(19) United States
(12) Patent Application Publication
(10) Pub. No.: US 2019/0060381 A1
(43) Pub. Date: Feb. 28, 2019

(54) CANNABINOID COMPOSITIONS, METHODS OF MANUFACTURE AND USE THEREOF

(71) Applicant: CANNABICS PHARMACEUTICALS INC., Bethesda, MD (US)

(72) Inventors: Eyal BALLAN, Ramat Hasharon (IL); Itamar BOROCHOV, Caesarea (IL)

(21) Appl. No.: 16/079,358

(22) PCT Filed: Feb. 23, 2017
(86) PCT No.: PCT/IL2017/050231

§ 371 Date: Aug. 23, 2018

Related U.S. Application Data
(60) Provisional application No. 62/299,011, filed on Feb. 24, 2016.

(30) Foreign Application Priority Data
Feb. 24, 2016 (IL) 244278

(51) Int. Cl. A61K 36/185; A61P 35/00; A61P 3/04; A61K 9/48; A61K 9/00

(52) U.S. Cl. CPC A61K 36/185; A61P 35/00; A61K 9/0056; A61K 9/4858; A61K 9/4833; A61P 3/04

(57) ABSTRACT

Provided are oral pharmaceutical compositions comprising sustained release or a combination of sustained and immediate release formulation of cannabinoids, a process for their preparation and methods of use thereof.

### FIELD OF THE INVENTION

The present disclosure relates to oral pharmaceutical compositions comprising sustained release or a combination of sustained and immediate release formulation of cannabinoids, a process for their preparation and methods of use thereof.

### BACKGROUND OF THE INVENTION

Cannabis has been reported to benefit patients suffering from a wide range of symptoms experienced in connection with serious medical conditions. For example, cannabis has been used to alleviate symptoms associated with cancer, anorexia, AIDS, chronic pain, muscle spasticity, glaucoma, arthritis, migraine and many other illnesses. Cannabis is recognized as having anti-emetic properties and has been successfully used to treat nausea and vomiting in cancer patients undergoing chemotherapy. Studies also report use of cannabis in treating the weight loss syndrome of AIDS and for the treatment of glaucoma by reducing intraocular pressure. Furthermore, cannabis is known for its muscle relaxing and anti-convulsant effects.

The most prevalent mode of administration of medical cannabis is by smoking. Unfortunately, this mode of administration has adverse effects on the lungs. Cannabis smoke carries more tar and other particulate matter than tobacco, and may be a cause of lung diseases including lung cancer. Furthermore, many patients find the act of smoking unappealing, as well as generally unhealthy. It is known that some of the chemicals produced by smoking cannabis are aggressive and smoking has been shown to cause the gradual dissolving of teeth. For at least these reasons, smoking is a less desirable mode of administration for drugs, including cannabis.

Cachexia

Cachexia originates from Greek and Latin roots: Kakos- (bad) and -hexis (condition or appearance). It is associated with several chronic diseases and, generally, involves a dual mechanism of general muscle wasting, malnutrition, and anorexia. Cachexia is primarily caused by cytokines released from inflammatory cells. In 2011, an international panel defined cachexia as a “multifactorial syndrome characterized by an ongoing loss of skeletal muscle mass (with or without loss of fat mass) that cannot be fully reversed by conventional nutritional support and leads to progressive functional impairment”.

Cachexia may be masked by excess weight, obesity, edema or tumor mass. Muscle wasting or sarcopenia occurs as a key feature of cachexia. In cachexia, muscle wasting is primarily caused by inflammation, in contrast to sarcopenia where muscle wasting is related to age and immobility. Additional parameters utilized to diagnose cachexia include food intake, CRP, and albumin levels.

Anorexia and Cachexia

Anorexia is a subjective term describing the reduction or loss of appetite. Although it is commonly known that patients suffering from cancer and cancer treatments experience loss of appetite, the exact prevalence of anorexia is unknown. In one study (n=351) on advanced cancer patients, more than half the patients experienced anorexia.

In patients suffering from cancer, anorexia may be due to physiological and psychological factors. Anorexia can occur due to chemotherapy treatments, which cause nausea and vomiting.

Tumors may also obstruct the upper gastrointestinal system, causing dysphagia and making it difficult to consume food. Depression also significantly contributes to decreased appetite. The decreased appetite causes increased psychological distress and a decreased quality of life.

The presence of a decreased appetite has been proposed to be an independent risk factor for mortality. A North Central Cancer Treatment Group study of 1,115 patients with colorectal and lung cancer found that patients with anorexia had lower survival rates and experienced more toxicity from chemotherapy than similarly matched patients who maintained their appetite.

In a retrospective review of 3,047 patients by the Eastern Cooperative Oncology group, weightloss greater than 5% before chemotherapy predicated early mortality regardless of stage or tumor type.

Cancer-Related Cachexia and Anorexia Syndrome

Cachexia may occur with or without a loss of appetite or reduction in nutrition. Interestingly, anorexia or decreased appetite is an independent risk factor for patient decline and, therefore, it has been proposed to view anorexia accompanying cachexia as a separate syndrome.

Cachexia primarily caused by anorexia or reduced intake has been defined as cancer-related cachexia and anorexia syndrome (CACS). CACS, unlike cachexia, includes weight-loss caused by muscle wasting, as well as lipolysis and decreased intake.

Pathogenesis of CACS

Anorexia is due to both catabolic drivers and inflammation associated with cancer, side effects associated with chemotherapy and radiation, as well as depression and other psychosocial effects.

It has been proposed that pro-inflammatory cytokines, such as IL-1, IL-6 and TNF-alpha, may decrease leptin release by mimicking excessive negative feedback signaling from leptin. In addition, these cytokines also contribute to hyper-metabolism and increased resting energy expenditure, especially as patients near death.

The symptoms correlating with decreased appetite in cancer have been referred to as “secondary nutrition impact symptoms” (S-NIS), and include early satiety, constipation, nausea or vomiting, dysphagia and depression. Increasing protein and caloric intake, may increase body mass and prolong survival. In order to have this effect, it has been suggested that patients should increase caloric intake by 300-400 kcal and protein intake by 50%. This was demonstrated in a study using parenteral nutrition, as meeting these goals may be difficult in patients experiencing S-NIS.

Decreased appetite may also lead to malnutrition. Although the relationship between malnutrition and cachexia is not well defined, it is proposed that a lack of certain nutrients further contributes to muscle catabolism, especially a deficiency of long chain n-3 polyunsaturated fatty acids, vitamin D, and choline. Treatments that address CACS should improve appetite in order to correct malnutrition associated with decreased protein and caloric intake.

Therapeutic Potential of Cannabinoid for CACS

Cannabis has long been suggested to stimulate appetite, decrease nausea and vomiting, and improve quality of life in cancer patients. Studies on the efficacy of cannabis for improving CACS and S-NIS have had mixed results. After trials showing improvement in weight gain among AIDS patients, cannabinoids were tested on cancer patients as well. In a controlled, random study comparing dronabinol to a placebo among cancer patients, dronabinol was associated with increased appetite in 38% vs 8% for placebo, and decreased nausea in 20% vs 7%, using acceptable measurement scales. Of the dronabinol patients, 22% gained ≥2 kg, compared with 10.5% of placebo recipients, but this datum did not reach significance, perhaps due to the advanced stage of cancer and the high mortality in both placebo and experimental group.

Another randomized study compared dronabinol to megestrol acetate or both treatments together.

The research included 469 advanced cancer patients who had been suffering from a substantial appetite loss. A greater percentage of megestrol acetate-treated patients reported appetite improvement compared with dronabinol-treated patients, 75% vs 49% (p=0.0001). Combination treatment resulted in no significant differences compared with megestrol acetate alone.

Another study, which included 243 patients, compared the administration of a combination of tetrahydrocannabinol and cannabinol to tetrahydrocannabinol alone, compared to placebo. It should be noted that cannabinoid dosages in the study were low, even in comparison to other studies. No significant differences between the groups were seen regarding improvement in appetite or weight-gain. In these two studies, no substantial side effects of cannabis products were found compared to the other arms. This may be related to the dosages of the drugs given.

A more recent study demonstrated improved chemosensory perception, appetite, sleep, and macronutrient preference in advanced cancer patients. However, the study included less than 50 patients. This research showed improvement in taste and smell perception in patients receiving chemotherapy, as well as appetite and caloric intake in the arm that received dronabinol compared to placebo.

Anti-Inflammatory Properties of Cannabis

Over 50 compounds have been isolated from cannabis. Generally, cannabinoids refer to compounds that activate CB1 and CB2 or have structures similar to delta-9-tetrahydrocannabinol (THC). THC has been found to reduce inflammation in chronic inflammatory diseases, such as atherosclerosis and rheumatoid arthritis. Cannabidiol (CBD), the most abundant nonpsychoactive cannabinoid, has also been studied for its anti-inflammatory properties. In synovial cells isolated from mice, CBD suppressed release of TNF-alpha. Cannaflavin A is 30× more potent than aspirin as an inhibitor of prostaglandin E2.

There is some evidence that PGE2 is involved in cachexia, and the reduction of prostaglandin E2 via inhibition of COX-2 has been shown to improve lean muscle mass. Beta-caryophyllene and luteolin are two nonpsychoactive, anti-inflammatory compounds found in cannabis and in other plants. Beta caryophyllene binds to CB2 and inhibits TNF alpha and IL-1b expression in human peripheral blood. Luteolin, a flavonoid found in celery and green pepper, has been found to suppress the production of TNFa, IL-1b, and IL-6 when added to peripheral blood mononuclear cells in vitro.

There remains an unmet need for a measurable, reproducible oral dosage form of cannabinoid for the treatment of multiple clinical conditions.

### SUMMARY OF THE INVENTION

It is thus one object of the present invention to provide an oral composition for the treatment of cancer-related cachexia and anorexia syndrome (CACS), the composition comprising at least one first cannabinoid compound and at least one second cannabinoid compound the at least one first cannabinoid compound is in an immediate release formulation and the at least one second cannabinoid compound is in a sustained-release formulation, wherein the first cannabinoid is Tetrahydrocannabinol (THC) and the second cannabinoid is Cannabidiol (CBD) and the ratio of the THC:CBD is about 95:0.5% w/w.

It is a further object of the present invention to provide the composition as defined above, wherein the ratio is selected from the group consisting of 90:10, 85:15, 80:20% w/w.

It is a further object of the present invention to provide the composition as defined in any of the above, wherein the cannabinoid compound comprises a lipophilic mixture of cannabinoids containing at least one cannabinoid selected from the group consisting of: Tetrahydrocannabinol (THC), Cannabidiol (CBD), Cannabigerol (CBG), Cannabichromene (CBC), Cannabinol (CBN), Cannabielsoin (CBE), iso-Tetrahydrocannabimol (iso-THC), Cannabicyclol (CBL), Cannabicitran (CBT), Cannabivarin (CBV), Tetrahydrocannabivarin (THCV), Cannabidivarin (CBDV), Cannabichromevarin (CBCV), Cannabigerovarin (CBGV) and Cannabigerol Monomethyl Ether (CBGM) and derivatives thereof.

It is a further object of the present invention to provide the composition as defined in any of the above, wherein the immediate release formulation comprises an edible oil and the sustained release formulation comprises at least one Lipid-based Drug Delivery System (LBDDS) agent.

It is a further object of the present invention to provide the composition as defined in any of the above, wherein the LBDDS agent is selected from the group consisting of a monoglyceride, a diglyceride, a carrageenan and any mixture thereof.

It is a further object of the present invention to provide the composition as defined in any of the above, wherein the LBDDS agent comprises a mixture of monoglycerides and diglycerides.

It is a further object of the present invention to provide the composition as defined in any of the above, wherein the LBDDS agent comprises a carrageenan.

It is a further object of the present invention to provide the composition as defined in any of the above, wherein the carrageenan is selected from the group consisting of lambda-carrageenan, kappa-carrageenan, iota-carrageenan and any mixture of the carrageenan.

It is a further object of the present invention to provide the composition as defined in any of the above, wherein the sustained release formulation further comprises an edible oil.

It is a further object of the present invention to provide the composition as defined in any of the above, wherein the edible oil in the immediate release formulation and in the sustained release formulation is independently selected from the group consisting of coconut oil, wheat sprout oil, olive oil, sprouted wheat oil, sesame oil, peanut oil, grape seed oil, palm oil, papaya seed oil or any combination thereof.

It is a further object of the present invention to provide the composition as defined in any of the above, wherein the edible oil in the immediate release formulation and in the sustained release formulation comprises coconut oil.

It is a further object of the present invention to provide the composition as defined in any of the above, wherein the composition comprises cannabis extract in a relative amount range of about 0.01 V/V to about 0.02 V/V.

It is a further object of the present invention to provide the composition as defined in any of the above, wherein the composition comprises Carrageenan in a relative amount of about 0.005 V/V.

It is a further object of the present invention to provide the composition as defined in any of the above, wherein the composition comprises Mono and Diglyceride in a relative amount of about 0.075 V/V.

It is a further object of the present invention to provide the composition as defined in any of the above, wherein the composition comprises coconut oil in a relative amount of about 0.92 V/V.

It is a further object of the present invention to provide the composition as defined in any of the above, further comprising at least one excipient selected from the group consisting of a diluent, a binder, a lubricant, a disintegrant, a flavoring agent, a coloring agent, a stabilizer, a surfactant, a glidant, a plasticizer, a preservative, an essential oil and a sweetener.

It is a further object of the present invention to provide the composition as defined in any of the above, wherein the therapeutic effect of the composition has a duration from 4 to 18 hours.

It is a further object of the present invention to provide the composition as defined in any of the above, wherein the composition comprising cannabis extract in a relative amount of about 0.01 V/V, Carrageenan in a relative amount of about 0.005 V/V, Mono and Diglyceride in a relative amount of about 0.075 V/V and coconut oil in a relative amount of about 0.92 V/V.

It is a further object of the present invention to provide the composition as defined in any of the above, wherein the cannabinoids are present in a total amount 2.5 mg to 50 mg per dosage form, preferably 5 mg per dosage form.

It is a further object of the present invention to provide the composition as defined in any of the above, wherein the dosage form is administered once or twice times per day, for a period of 3 days to 6 months.

It is a further object of the present invention to provide the composition as defined in any of the above, wherein the syndrome is associated with a symptom selected from the group consisting of: weight loss, appetite loss, reduced caloric intake, elevated TNF-alpha level, anorexia, cachexia, reduced Quality-of-Life, mood related conditions, gastrointestinal problems, reduced muscle mass, reduced muscle strength, pain, and any combination thereof.

It is a further object of the present invention to provide the composition as defined in any of the above, wherein the composition provides a beneficial effect selected from the group consisting of weight gain of at least 10% from baseline weight, improvement in appetite, improvement in caloric intake, reduction in TNF-alpha level, analgesic effects, antitumor activity, cancer cells cytotoxic effect, antidepressant, an anxiolytic, neuroprotective, anti-psychotic, improvement in quality of life (QoF) and any combination thereof.

It is a further object of the present invention to provide the composition as defined in any of the above, wherein the improvement in quality of life (QoF) is assessed using the European Organization of Research and Treatment of Cancer core questions on the Quality of Life Questionnaire, version 2 (QLQ-C30) and the Anorexia/Cachexia Therapy (FAACT) questionnaire.

It is a further object of the present invention to provide the composition as defined in any of the above, wherein the composition is formulated as granules, powder, capsules, tablet, film, suspension, sachets, a chewing gum and suspension.

It is a further object of the present invention to provide the composition as defined in any of the above, wherein the composition is formulated as a gelatin capsule.

It is a further object of the present invention to provide a method for the treatment of cancer-related cachexia and anorexia syndrome (CACS) in a subject, comprising steps of: (a) providing a composition comprising at least one first cannabinoid compound and at least one second cannabinoid compound, wherein the at least one first cannabinoid compound is in an immediate release formulation and the at least one second cannabinoid compound is in a sustained-release formulation; and (b) administering the composition to the subject orally in a therapeutically effective dosage form.

It is a further object of the present invention to provide the method as defined above, comprising steps of providing an oral composition comprising at least one first cannabinoid compound and at least one second cannabinoid compound the at least one first cannabinoid compound is in an immediate release formulation and the at least one second cannabinoid compound is in a sustained-release formulation, wherein the first cannabinoid is Tetrahydrocannabinol (THC) and the second cannabinoid is Cannabidiol (CBD) and the ratio of the THC:CBD is about 95:0.5% w/w.

It is a further object of the present invention to provide the method as defined in any of the above, additionally comprising steps of administering the composition in a dosage form comprising cannabinoids in a total amount of 2.5 mg to 50 mg per dosage form, preferably 5 mg per dosage form.

It is a further object of the present invention to provide the method as defined in any of the above, additionally comprising steps of administering the dosage form once or twice times per day, for a period of 3 days to 6 months.

It is a further object of the present invention to provide the method as defined in any of the above, further comprising steps of treating a symptom associated with the syndrome, the symptom is selected from the group consisting of: weight loss, appetite loss, reduced caloric intake, elevated TNF-alpha level, anorexia, cachexia, reduced Quality-of-Life, mood related conditions, gastrointestinal problems, reduced muscle mass, pain, and any combination thereof.

It is a further object of the present invention to provide the method as defined in any of the above, further comprising steps of providing a beneficial effect selected from the group consisting of weight gain of at least 10% from baseline weight, improvement in appetite, improvement in caloric intake, reduction in TNF-alpha level, analgesic effects, antitumor activity, cancer cells cytotoxic effect, antidepressant, an anxiolytic, neuroprotective, anti-psychotic, improved quality of life, and any combination thereof.

It is a further object of the present invention to provide the method as defined in any of the above, wherein the improvement in quality of life is assessed using at least one of the European Organization of Research and Treatment of Cancer core questions on the Quality of Life Questionnaire, version 2 (QLQ-C30) and the Anorexia/Cachexia Therapy (FAACT) questionnaire.

It is a further object of the present invention to provide the method as defined in any of the above, further comprising steps of attenuating a symptom of the syndrome, treat the syndrome or attenuating a side effect of a treatment of the syndrome.

It is a further object of the present invention to provide the method as defined in any of the above, wherein the method exerts reduced hallucinatory effects in the subject when compared to smoking a cannabis containing cigarette or ingesting a cannabis containing foodstuff with the same amount of active ingredient.

It is a further object of the present invention to provide the method as defined in any of the above, additionally comprising steps of administering the dosage form once per day for the first 3 to 4 days of the treatment; and twice per day from the 5th day of the treatment.

It is a further object of the present invention to provide a process for the preparation of the composition of claim 1, comprising the steps of (a) mixing a cannabinoid extract with an edible oil to formulate an immediate release (IR) formulation; (b) filling a capsule with the IR formulation; (c) freezing the filled capsule at −20° C.; (d) thawing the freezed capsule; (e) mixing a cannabinoid extract with at least one Lipid-based Drug Delivery System (LBDDS) agent and optionally an edible oil, to formulate sustained release (SR) formulation; and (f) adding the sustained release (SR) formulation to the thawed capsule.

It is a further object of the present invention to provide the process as defined above further comprising steps of adding at least one of Monoglycerides, Diglycerides and Carrageenan to the mixture of step e).

It is a further object of the present invention to provide the process as defined in any of the above further comprising steps of milling, drying, compressing or filling capsules, preferably filling capsules.

It is a further object of the present invention to provide the process as defined in any of the above, further comprising steps of mixing a cannabinoid extract comprising 5 mg of cannabinoids.

It is a further object of the present invention to provide a use of a composition comprising at least one first cannabinoid compound and at least one second cannabinoid compound, wherein the at least one first cannabinoid compound is in an immediate release formulation and the at least one second cannabinoid compound is in a sustained-release formulation in the manufacture of a medicament for treating cancer-related cachexia and anorexia syndrome (CACS) in a subject.

It is a further object of the present invention to provide the use as defined above, additionally comprising steps of administering the composition to the subject orally in a therapeutically effective dosage form.

It is a further object of the present invention to provide the use as defined in any of the above, comprising steps of providing an oral composition comprising at least one first cannabinoid compound and at least one second cannabinoid compound the at least one first cannabinoid compound is in an immediate release formulation and the at least one second cannabinoid compound is in a sustained-release formulation, wherein the first cannabinoid is Tetrahydrocannabinol (THC) and the second cannabinoid is Cannabidiol (CBD) and the ratio of the THC:CBD is about 95:0.5% w/w.

It is a further object of the present invention to provide the use as defined in any of the above, additionally comprising steps of administering the composition in a dosage form comprising cannabinoids in a total amount of 2.5 mg to 50 mg per dosage form, preferably 5 mg per dosage form.

It is a further object of the present invention to provide the use as defined in any of the above, additionally comprising steps of administering the dosage form once or twice times per day, for a period of 3 days to 6 months.

It is a further object of the present invention to provide the use as defined in any of the above, further comprising steps of treating a symptom associated with the syndrome, the symptom is selected from the group consisting of: weight loss, appetite loss, reduced caloric intake, elevated TNF-alpha level, anorexia, cachexia, reduced Quality-of-Life, mood related conditions, gastrointestinal problems, reduced muscle mass, pain, and any combination thereof.

It is a further object of the present invention to provide the use as defined in any of the above, further comprising steps of providing a beneficial effect selected from the group consisting of weight gain of at least 10% from baseline weight, improvement in appetite, improvement in caloric intake, reduction in TNF-alpha level, analgesic effects, antitumor activity, cancer cells cytotoxic effect, antidepressant, an anxiolytic, neuroprotective, anti-psychotic, improved quality of life, and any combination thereof.

It is a further object of the present invention to provide the use as defined in any of the above, wherein the improvement in quality of life is assessed using at least one of the European Organization of Research and Treatment of Cancer core questions on the Quality of Life Questionnaire, version 2 (QLQ-C30) and the Anorexia/Cachexia Therapy (FAACT) questionnaire.

It is a further object of the present invention to provide the use as defined in any of the above, further comprising steps of attenuating a symptom of the syndrome, treat the syndrome or attenuating a side effect of a treatment of the syndrome.

It is a further object of the present invention to provide the use as defined in any of the above, wherein the use exerts reduced hallucinatory effects in the subject when compared to smoking a cannabis containing cigarette or ingesting a cannabis containing foodstuff with the same amount of active ingredient.

It is a further object of the present invention to provide the use as defined in any of the above, additionally comprising steps of administering the dosage form once per day for the first 3 to 4 days of the treatment; and twice per day from the 5th day of the treatment.

It is a further object of the present invention to provide a kit useful for treating cancer-related cachexia and anorexia syndrome (CACS) comprising: (a) a plurality of orally administrable dosage forms, wherein each dosage form comprising a composition comprising at least one first cannabinoid compound and at least one second cannabinoid compound the at least one first cannabinoid compound is in an immediate release formulation and the at least one second cannabinoid compound is in a sustained-release formulation, wherein the first cannabinoid is Tetrahydrocannabinol (THC) and the second cannabinoid is Cannabidiol (CBD) and the ratio of the THC:CBD is about 95:0.5% w/w; and instructions for use of the dosage forms.

It is a further object of the present invention to provide the kit as defined above, wherein the ratio is selected from the group consisting of 90:10, 85:15, 80:20% w/w.

It is a further object of the present invention to provide the kit as defined in any of the above, wherein the cannabinoid compound comprises a lipophilic mixture of cannabinoids containing at least one cannabinoid selected from the group consisting of: Tetrahydrocannabinol (THC), Cannabidiol (CBD), Cannabigerol (CBG), Cannabichromene (CBC), Cannabinol (CBN), Cannabielsoin (CBE), iso-Tetrahydrocannabimol (iso-THC), Cannabicyclol (CBL), Cannabicitran (CBT), Cannabivarin (CBV), Tetrahydrocannabivarin (THCV), Cannabidivarin (CBDV), Cannabichromevarin (CBCV), Cannabigerovarin (CBGV) and Cannabigerol Monomethyl Ether (CBGM) and derivatives thereof.

It is a further object of the present invention to provide the kit as defined in any of the above, wherein the immediate release formulation comprises an edible oil and the sustained release formulation comprises at least one Lipid-based Drug Delivery System (LBDDS) agent.

It is a further object of the present invention to provide the kit as defined in any of the above, wherein the LBDDS agent is selected from the group consisting of a monoglyceride, a diglyceride, a carrageenan and any mixture thereof.

It is a further object of the present invention to provide the kit as defined in any of the above, wherein the LBDDS agent comprises a mixture of monoglycerides and diglycerides.

It is a further object of the present invention to provide the kit as defined in any of the above, wherein the LBDDS agent comprises a carrageenan.

It is a further object of the present invention to provide the kit as defined in any of the above, wherein the carrageenan is selected from the group consisting of lambda-carrageenan, kappa-carrageenan, iota-carrageenan and any mixture of the carrageenan.

It is a further object of the present invention to provide the kit as defined in any of the above, wherein the sustained release formulation further comprises an edible oil.

It is a further object of the present invention to provide the kit as defined in any of the above, wherein the edible oil in the immediate release formulation and in the sustained release formulation is independently selected from the group consisting of coconut oil, wheat sprout oil, olive oil, sprouted wheat oil, sesame oil, peanut oil, grape seed oil, palm oil, papaya seed oil or any combination thereof.

It is a further object of the present invention to provide the kit as defined in any of the above, wherein the edible oil in the immediate release formulation and in the sustained release formulation comprises coconut oil.

It is a further object of the present invention to provide the kit as defined in any of the above, wherein the composition comprises cannabis extract in a relative amount range of about 0.01 V/V to about 0.02 V/V.

It is a further object of the present invention to provide the kit as defined in any of the above, wherein the composition comprises Carrageenan in a relative amount of about 0.005 V/V.

It is a further object of the present invention to provide the kit as defined in any of the above, wherein the composition comprises Mono and Diglyceride in a relative amount of about 0.075 V/V.

It is a further object of the present invention to provide the kit as defined in any of the above, wherein the composition comprises coconut oil in a relative amount of about 0.92 V/V.

It is a further object of the present invention to provide the kit as defined in any of the above, further comprising at least one excipient selected from the group consisting of a diluent, a binder, a lubricant, a disintegrant, a flavoring agent, a coloring agent, a stabilizer, a surfactant, a glidant, a plasticizer, a preservative, an essential oil and a sweetener.

It is a further object of the present invention to provide the kit as defined in any of the above, wherein the therapeutic effect of the composition has a duration from 4 to 18 hours.

It is a further object of the present invention to provide the kit as defined in any of the above, wherein the composition comprising cannabis extract in a relative amount of about 0.01 V/V, Carrageenan in a relative amount of about 0.005 V/V, Mono and Diglyceride in a relative amount of about 0.075 V/V and coconut oil in a relative amount of about 0.92 V/V.

It is a further object of the present invention to provide the kit as defined in any of the above, wherein the cannabinoids are present in a total amount 2.5 mg to 50 mg per dosage form, preferably 5 mg per dosage form.

It is a further object of the present invention to provide the kit as defined in any of the above, wherein the dosage form is administered once or twice times per day, for a period of 3 days to 6 months.

It is a further object of the present invention to provide the kit as defined in any of the above, wherein the syndrome is associated with a symptom selected from the group consisting of: weight loss, appetite loss, reduced caloric intake, elevated TNF-alpha level, anorexia, cachexia, reduced Quality-of-Life, mood related conditions, gastrointestinal problems, reduced muscle mass, reduced muscle strength, pain, and any combination thereof.

It is a further object of the present invention to provide the kit as defined in any of the above, wherein the composition provides a beneficial effect selected from the group consisting of weight gain of at least 10% from baseline weight, improvement in appetite, improvement in caloric intake, reduction in TNF-alpha level, analgesic effects, antitumor activity, cancer cells cytotoxic effect, antidepressant, an anxiolytic, neuroprotective, anti-psychotic, improvement in quality of life (QoF) and any combination thereof.

It is a further object of the present invention to provide the kit as defined in any of the above, wherein the improvement in quality of life (QoF) is assessed using the European Organization of Research and Treatment of Cancer core questions on the Quality of Life Questionnaire, version 2 (QLQ-C30) and the Anorexia/Cachexia Therapy (FAACT) questionnaire.

It is a further object of the present invention to provide the kit as defined in any of the above, wherein the composition is formulated as granules, powder, capsules, tablet, film, suspension, sachets, a chewing gum and suspension.

It is a further object of the present invention to provide the kit as defined in any of the above, wherein the dosage form is formulated as a gelatin capsule.

### BRIEF DESCRIPTION OF THE FIGURES

FIG. 1 shows a schematic flowchart of the manufacturing process overview; and

FIG. 2 shows a schematic representation of the time-dependent release effect of THC and CBD of exemplified composition of the present invention.

### DETAILED DESCRIPTION OF THE PREFERRED EMBODIMENTS

The preferred methods, uses, materials, and examples that will now be described are illustrative only and are not intended to be limiting; materials, uses and methods similar or equivalent to those described herein can be used in practice or testing of the invention. Other features and advantages of the invention will be apparent from the following figures, detailed description, and from the claims.

The present invention provides an oral composition for the treatment of disease symptoms, preferably symptoms of cachexia and anorexia, said composition comprising at least one first cannabinoid compound and at least one second cannabinoid compound, wherein said at least one first cannabinoid compound is in an immediate release formulation and said at least one second cannabinoid compound is in a sustained-release formulation.

The compositions of the present invention are formulated and designed for providing an improvement of at least one disease symptom, especially cancer related cachexia and anorexia syndrome (CACS), including weight loss, appetite loss, reduced caloric intake, elevated TNF-alpha level, reduced Quality-of-Life, mood related conditions, gastrointestinal problems, reduced muscle mass, pain, and any combination thereof or other syndrome related to CACS.

According to one embodiment, the therapeutic compositions of the present invention are useful for treating CACS. Formulations within the scope of the present invention may comprise sustained release or combined immediate and sustained release cannabinoid formulations or fractions. The sustained release fraction comprises an active pharmaceutical ingredient (i.e. cannabinoid extract or synthetic cannabinoid) such as THC and an emulsifier. Non limiting examples of emulsifiers used in the formulation of the present invention include monoglyeride, diglyceride, carrageenan Lota/Kappa, or any mixture thereof, and optionally an edible oil or a mixture of edible oils. The advantages of the composition over known cannabis compositions are manifold and include:

(a) High bioavailability of the APIs in the composition;

(b) A flat phamacokinetic profile that enables steady state level of beneficial effects for a duration of at least 4-10 hours.

(c) Avoidance of a sharp Cmax of APIs in the circulation and thus reduced level of undesirable side effects, or in other words, a lower peak of the psychoactive effect with a longer therapeutic window and lower Cmax.

(d) Once to twice per day easy administration regimen that promotes high patient compliance.

The current invention discloses cannabinoid formulation delivered in a slow release (SR) and/or immediate release (IR) i.e. comprising oils, glycerides and carrageenan.

The formulations of the present invention are preferably directed to oral administration of cannabinoids. The SR and IR formulations deliver different cannabinoids in a gradual manner to provide therapeutic effects. Without wishing to be bound by theory, it is noted that since cannabinoids are similar molecules with common receptors, mainly, CB1 and CB2, they are both competitors and adjuvants. The entourage effect is based on that concept thus enabling the control of the desired immediate effects and prolonged effects that are related to the different cannabinoids. For example: for reducing the psychoactive effect of THC, yet maintaining high levels of it in the blood for a longer effect, a rich CBD extract may be used in a IR formulation and THC rich extract in SR formulation.

It is within the scope that the composition of the present invention comprises varied levels of SR (% of oils and glycerides and carrageenan) and IR formulation, combined with:

1. Natural enriched extracts (such as high CBD/THC/CBN cannabis strains)

2. Synthetic cannabinoids

3. Both synthetic and natural cannabinoids

4. Natural or synthetic cannabinoid combined with synergistic compounds such as cocoa butter

Reference is Now Made to the Following Aspects of the Invention:

Cannabis is a medicinal plant, known to humankind for centuries. Since 1920 until recent years, scientific research and development was halted due to regulation created by economic interests. In recent years, a growing number of states remove regulation constrains enabling researchers to study the medicinal properties of cannabis and improve its administration to patients.

It is herein acknowledged that Cannabis has several beneficial effects. Cannabinoids can serve as appetite stimulants, antiemetic, antispasmodics, and have an analgesic effects thus being a potential medicine for cancer patients. Millions of patients could benefit from its therapeutic properties and the unmet need of these patients is a safe, standardized and easy to administer cannabis-based therapy.

The cannabis capsules and therapy of the present invention have been developed to answer these unmet needs, mainly to create once to twice-daily regimen of a standardized natural cannabis therapy. The main property of the cannabis capsules of the present invention is its Lipid-based Drug Delivery System (LBDDS) formulation that enables a prolonged therapeutic window observed in preliminary POC (proof of concept) study and it is based solely on food-grade ingredients.

According to one embodiment, the cannabis capsules of the present invention are designed as a treatment to improve Cancer Related Cachexia and Anorexia Syndrome in advanced cancer patients. The disclosed cannabis therapy provides an improvement in at least one of the following: weight gain, improvement in appetite and caloric intake, and safety, thus establishing a palliative treatment.

In the present invention, a correlation between treatment with cannabis formulation and levels of TNF-alpha 1 is demonstrated as a marker of a change in cancer cell vitality and tumor progression.

According to a further aspect, the cannabis formulation and treatment is shown to be effective for the treatment of chronic pain, including pain caused by neuropathy and also to fibromyalgia and rheumatoid arthritis.

With respect to neurological conditions and disorders, the cannabis formulation and treatment of the present invention is effective in multiple sclerosis, epilepsy, and movement problems. The combined potential of a wide therapeutic spectrum and low toxicity makes the herein disclosed cannabis formulation a highly effective medicine.

As used herein, the term “about” refers hereinafter to ±25% of the defined amount or measure or value.

As used herein, the term “treatment” refers to therapeutic treatment of cannabinoid responsive disorder, wherein the object of the treatment is to reduce or reverse the symptoms of the disorder. In preferred embodiments the present invention provides cannabis capsules as treatment to cancer related cachexia and anorexia syndrome in advanced cancer patients. Those in need of treatment include those already experiencing the disease or condition, for example, pain, weight loss and reduced appetite in cancer patients or nausea in chemotherapy patients. The compositions or combinations disclosed herein are administered during or subsequent to the onset of the disease, symptom, syndrome or condition. In some embodiments, treatment refers to prophylaxis, for example, prophylaxis of a disorder in a subject at risk of developing such a disorder, such as nausea in chemotherapy patients. Cₘₐₓ refers to the maximum (or peak) concentration (for example in the blood stream) that a drug achieves after the drug has been administered.

In some embodiments, a cannabinoid responsive disorder is selected from disorders responsive to treatment with cannabis including but not limited to cancer-related cachexia and anorexia syndrome (CACS).

The term “immediate release formulation” or IR formulation as used herein refers to a dosage form comprising cannabis extract or cannabinoid extract mixed with an edible oil, responsible for the quick onset of the therapeutic effects within about 20-60 minutes.

The term “slow release formulation” or SR formulation as used herein refers to a dosage for comprising cannabis extract or cannabinoid extract mixed with at least one Lipid-based Drug Delivery System (LBDDS) agent, also referred to as an emulsifier. Examples of an LBDDS agent used in the present invention include monoglyceride, diglyceride (E471) and carrageenan. The mixture of cannabis extract with LBDDS formulate a consolidated Cannabinoid-LBDDS fraction, which is responsible for a gradual and long lasting therapeutic effect (about 6-8 hr) due to a proposed constant and steady release of active cannabinoids. The formulation contains extract of cannabinoids, monoglyceride and diglyceride (E471), combined with carrageenan which is known for its controlled release properties and optionally organic coconut oil.

The therapeutic window refers to the range of drug dosages of a medication that elicit a therapeutic response, without unacceptable adverse effects (toxicity), in a population of patients. Therapeutic window may also be referred to as “effective dose”. The therapeutic window of the present composition per dose form is about 1 mg to about 350 mg cannabinoid, particularly, about 5 mg to about 250 mg and more particularly, about 5 mg to about 100 mg.

According to a specific embodiment, the composition of the present invention comprises between about 2.5 mg to about 30 mg, preferably 5 mg of API cannabinoid compound.

In some embodiments, each dosage form of the composition of the present invention comprises about 9.5 mg of Tetrahydrocannabinol (THC) in a sustained release formulation and about 0.5 mg of Cannabidiol (CBD) in an immediate release formulation.

In other embodiments, each dosage form of the composition of the present invention comprises about 4.75 mg of Tetrahydrocannabinol (THC) in a sustained release formulation and about 0.25 mg of Cannabidiol (CBD) in an immediate release formulation.

In some embodiments, each dosage form contains, for example, relative amount of about 0.01 V/V to about 1.0 V/V, preferably about 0.01 V/V of cannabis extract.

In some embodiments, a dose unit includes 2.5 mg to 50 mg API i.e. cannabis extract or cannabinoids in a sustained release or a combination of sustained release and immediate release, 6 mg to 40 mg API or dose units of 5 mg, 6 mg, 12 mg, 25 mg and 40 mg, which corresponds to 1%, 1.2%, 2.4%, 5% and 8% API in the formulation, respectively. Each dosage form or unit, may contain from about 30 mg to about 500 mg, or about 100 to about 500 mg, or about 500 mg total composition, which includes sustained release or combined immediate release and sustained release fractions.

Pharmaceutical preparations for the combination therapy for oral, enteral or parenteral administration are, for example, those in unit dosage forms, such as sugar-coated tablets, tablets, capsules, gelatin capsules or suppositories, or ampoules. If not indicated otherwise, these are prepared in a manner known per se, for example by means of conventional mixing, granulating, coating, dissolving or lyophilizing processes.

It is within the scope that the unit content of API per fraction contained in an individual dosage form itself, constitute an effective amount.

In other embodiments, the necessary effective amount can be reached by administration of a plurality of dosage units.

In some embodiments, the dosage form is a capsule or tablet. Capsule formulations may be a hard gelatin or soft gelatin type that contains the active API in solid, semi-solid, or liquid form. Gelatin capsules are formed from animal gelatin or synthetic or plant derived equivalents thereof. In some embodiments, the oral compositions disclosed herein are contained in a soft, vegetarian gelatin capsule.

As used herein, the singular forms “a”, “an” and “the” include plural forms unless the content clearly dictates otherwise.

The term “Cannabinoid” or “cannabinoid compound” as used herein refers to the compositions disclosed herein providing one or more cannabinoids in an oral dosage form that can deliver to a subject a desired target PK profile, where the PK profile achieves a therapeutic level of active cannabinoids within a therapeutic window. Cannabinoids useful in the compositions disclosed are any member of a group of substances or compounds that bind to a cannabinoid receptor such as CB1 or CB2 or both. The cannabinoid can be a naturally occurring compound (e.g. present in Cannabis), a compound metabolized by a plant or animal, or a synthetic derivative. It is within the scope that the cannabinoid compound may include endocannabinoids (produced naturally in the body by humans and animals), phytocannabinoids (found in cannabis and some other plants), and synthetic cannabinoids (manufactured artificially). The cannabinoid may be included in its free form, or in the form of a salt; an acid addition salt of an ester; an amide; an enantiomer; an isomer; a tautomer; a prodrug; a derivative of an active agent of the present invention; different isomeric forms (for example, enantiomers and diastereoisomers), both in pure form and in admixture, including racemic mixtures; enol forms.

In some embodiments, the cannabinoid(s) utilized in the present invention are a lipophilic concentrate of active cannabinoids achieved via CO2 extraction technique, and represents only one example of the different forms and extraction methods of cannabinoids useful for preparing the compositions disclosed herein.

Cannabis sativa contains over 421 different chemical compounds, including over 60 cannabinoids. Eighteen different classes of chemicals, including nitrogenous compounds, amino acids, hydrocarbons, carbohydrates, terpenes, and simple and fatty acids, contribute to the known pharmacological and toxicological properties of cannabis.

The cannabinoids of the present invention can be any of a synthetic or natural 9-tetrahydrocannabinol (THC), 8-tetrahydrocannabinol, (+)-1,1-dimethylheptyl analog of 7-hydroxy-delta-6-tetrahydrocannabinol, cannabinol (CBN), cannabidivarin (CBDV), cannabidiolic acid (CBDA), cannabidiol (CBD), cannabichromene (CBC), cannabigerol (CBG), 3-(5′-cyano-1′,1′-dimethylpentyl)-1-(4-N-morpholinobutyryloxy) delta 8-tetrahydrocannabinol hydrochloride], dexanabinol, nabilone, levonantradol, or N-(2-hydroxyethyl)hexadecanoamide. The cannabinoids of the present invention can be any of the psychotropic or non-psychotropic cannabinoids. In preferred embodiments, the lipophilic mixture of cannabinoids comprises the following cannabinoid types and their derivatives (including their acidic and decarboxylated derivatives): Tetrahydrocannabinol (THC), Cannabidiol (CBD), Cannabigerol (CBG), Cannabichromene (CBC), Cannabinol (CBN), Cannabielsoin (CBE), iso-Tetrahydrocannabimol (iso-THC), Cannabicyclol (CBL), Cannabicitran (CBT), Cannabivarin (CBV), Tetrahydrocannabivarin (THCV), Cannabidivarin (CBDV), Cannabichromevarin (CBCV), Cannabigerovarin (CBGV) and Cannabigerol Monomethyl Ether (CBGM).

Suitable amounts of API, e.g. cannabis extract, may be introduced and these amounts can be empirically determined using standard methods. The weight ratio of the cannabinoid extract to the excipients mixture may range from 1% to 99% w/w. Effective concentrations of individual dosage forms may range from 1% to 20% w/w which reflects about 5 mg to 100 mg, respectively. In other embodiments, effective concentrations of individual dosage forms may range from 0.5% to 20% w/w which reflects about 2.5 mg to 100 mg, respectively.

In some embodiments, each dosage form contains, about 0.01 V/V to about 1.0 V/V, preferably about 0.01 V/V, of cannabis extract relative amount.

Route of Administration of Cannabinoids:

The unmet need of patients who could benefit from the therapeutic properties of cannabinoids is a safe, standardized and easy to administer cannabinoid-based therapy. While clinical studies show contradictive data regarding a correlation between smoking cannabis and respiratory diseases, most physicians agree that smoking medical cannabis, is not a healthy nor standardized therapy.

Children and the elderly cannot smoke and the majority of patients dislike the connotation of “smoking drugs” thus depriving themselves from the physical and mental therapeutic benefits.

According to one aspect, the pharmacokinetics of THC varies as a function of its route of administration. Pulmonary assimilation of inhaled THC causes a maximum plasma concentration within minutes; psychotropic effects start within seconds to a few minutes, reach a maximum after 15-30 minutes, and taper off within 2-3 hours. Following oral ingestion, psychotropic effects set in with a delay of 30-90 minutes, reach their maximum after 2-3 hours and last for about 4-12 hours, depending on dose and specific effect. Another route of administration is sublingually. Pure cannabinoids are extracted from the raw plant, dissolved in different oils and administered with a dropper. The therapeutic window of sublingual oil administration is 2-4 hours with a fast onset due to quick absorption through the oral cavity. The most common oral administration of cannabinoids is through eating edibles, mainly, cookies, chocolate bars and lozenges. Since absorption is attenuated when cannabinoids are ingested orally, edibles usually contain high dosages of cannabinoids (50-300 mg). The high dosage may cause undesirable side effects, mainly, dizziness, anxiety and dissociation. These side effects cause many patients to withdraw from the therapeutic process.

It is within the scope that the oral administration route has the longest therapeutic window (4-8 hours) and lacks the undesirable effects of smoking. The unmet need for an oral formulation with higher bioavailability and a lower peak of psychoactive effect led to the formulation of the present invention which is a new oral capsule that is a standardized with a longer therapeutic window and lower Cmax. The formulation of the capsule is Lipid-based drug delivery systems (LBDDS) which highly improves the relatively low oral bioavailability (related to absorption, degradation and metabolism). According to further main aspects of the invention, the cannabis capsule of the present invention is designed for providing improvement and treatment of cancer-related cachexia and anorexia syndrome (CACS).

Reference is Now Made to the Lipid-Based Formulations for Oral Delivery of Lipophilic Drugs:

Lipid-based formulations are herein shown to improve the biopharmaceutical performance of lipophilic drugs compared to a conventional dosage form. There is typically an increase of oral bioavailability, but other effects like better linearity of exposure or less variability within and between subjects may be observed as well. Lipid-based drug delivery systems (LBDDS) are used herein as a key technology to formulate lipophilic compounds.

According to some embodiments, the composition of the present invention comprises monoglyceride, which is a molecule with one glycerol moiety covalently bonded to a fatty acid chain via an ester bond.

According to other embodiments, the composition of the present invention comprises diglyceride, which is a molecule with one glycerol moiety covalently bonded to two fatty acid chains via ester bonds.

According to other embodiments, the composition of the present invention comprises a mixture of monoglyceride and diglyceride (Glice-E471), which act as a type I LBDDS.

In some embodiments, the composition of the present invention comprises an edible oil such as coconut oil. It is within the scope that the immediate release formulation fraction comprises the edible oil. In other embodiments of a combined immediate release and sustained release composition, both the immediate release fraction and the sustained release fraction comprise an edible oil.

In some embodiments, the oil is a vegetable, fruit, seed, nut or synthetic oil selected from coconut oil, wheat sprout oil, wheat germ oil, olive oil, sesame oil, peanut oil, almond oil, grape seed oil, palm oil, papaya seed oil, canola oil, sunflower oil, or a mixture thereof. The edible oil is preferably coconut oil or a mixture of coconut oil and another edible oil. Preferably, the edible oil is an organic edible oil, for example organic coconut oil and/or wheat sprout oil, for example organic wheat sprout oil.

According to one aspect, the composition of the present invention comprises coconut oil in a relative amount of about 0.9 V/V.

The compositions disclosed herein include at least one emulsifier. The preferred emulsifier is selected from the group consisting of a monoglyceride, a diglyceride, beeswax, lecithin, a carrageenan and any mixture thereof. In some embodiments the composition includes an emulsifier in a concentration of 1% to 99% w/w. In preferred embodiments, the composition comprises an emulsifier at a relative concentration range of about 0.005 V/V % to 1 V/V %, about 0.005 V/V % to 0.08 V/V % and about 0.01 V/V % to 0.075 V/V %.

In some embodiments, the emulsifier comprises a monoglyceride, a diglyceride or a mixture of a monoglyceride and a diglyceride. In some embodiments, the emulsifier comprises more than one monoglyceride and/or diglyceride.

The monoglyceride, diglyceride or mixture of monoglyceride and diglyceride act as an emulsifier. A preferred emulsifier known in the art as “Glice” or “E471” is a mixture of monoglycerides and diglycerides that has gelling properties when mixed with oil, and forms a butter-like oil-gel.

In some embodiments the composition comprises a monoglyceride, a diglyceride or a mixture of a monoglyceride and a diglyceride at a concentration of about 0.005 V/V % to 0.1 V/V %, about 0.075 V/V % to 0.01 V/V %.

In some embodiments, the emulsifier may comprise a polysaccharide. The polysaccharide may be linear or branched, sulfated or unsulfated. In some embodiments, the composition comprises one or more linear sulfated polysaccharide known as “carrageenan”.

The carrageenan is a family of linear sulfated polysaccharides that are extracted from edible seaweed and widely used in the food industry. The USPNF 23 describes carrageenan as hydrocolloid obtained by extraction and purification with water or aqueous alkali from few members of the class Rhodophyceae (red seaweed). It consists mainly of potassium, sodium, calcium magnesium and ammonium sulfate esters of galactose and 3,6-anhydrogalactose copolymers. These hexoses are alternatively linked at the α-1,3 and β-1,4 sites in the polymer.

The carrageenans are divided into three families according to the position of sulfate groups and the presence of anhydrogalactose. Lambda-carrageenan (λ-carrageenan) is a nongelling polymer containing about 35% ester sulfate by weight and no 3,6-anhydrogalactose. Iota-carrageenan (ι-carrageenan) is a gelling polymer containing about 32% ester sulfate by weight and approximately 30% 3,6-anhydrogalactose. Kappa carrageenan (κ-carrageenan) is a strongly gelling polymer which has a helical tertiary structure that allows gelling. It contains 25% ester sulfate by weight and approximately 34% 3,6-anhydrogalactose. Among the three carrageenans, λ-carrageenan is the only nongelling polymer.

In some embodiments the composition comprises a carrageenan or a mixture of carrageenans at a concentration of 0.005 V/V % to about 0.01V/V %, or about 0.01% to 10% w/w, preferably at a concentration of 0.01% to about 5% or 1% to about 7% w/w.

Therefore, an emulsifier selected from λ-carrageenan, κ-carrageenan, ι-carrageenan, monoglyceride, diglyceride, lecithin, beeswax or any mixture thereof, when formulated with at least one cannabinoid or cannabinoid extract, or cannabis oil extract and optionally an edible oil and/or one or more pharmaceutical excipient provides sustained or extended release of cannabinoids.

Depending on the dosage form, the one or more optional pharmaceutical excipient may be selected from a diluent, a binder, a lubricant, a disintegrant, a flavoring agent, a coloring agent, a stabilizer, a surfactant, a glidant, a plasticizer, a preservative, essential oil and a sweetener. A person skilled in the art will be able to select the best excipient or mixture of excipients for the desired formulation. Each excipient may fall within one or more classifications.

A diluent may be selected from, for example, calcium carbonate, calcium phosphate dibasic, calcium phosphate tribasic, calcium sulfate, microcrystalline cellulose, microcrystalline silicified cellulose, powdered cellulose, dextrate, dextrose, fructose, lactitol, lactose anhydrous, lactose monohydrate, lactose dihydrate, lactose trihydrate, mannitol, sorbitol, starch, pregelatinized starch, sucrose, talc, xylitol, maltose, maltodextrin, maltitol.

A binder may be selected from, for example, acacia, alginic acid, carbomer, carboxymethylcellulose calcium, carbomethylcellulose sodium, microcrystalline cellulose, powdered cellulose, ethyl cellulose, gelatin liquid glucose, guar gum, hydroxyethyl cellulose, hydroxypropyl cellulose, hydroxypropylmethyl cellulose, maltodextrin, methylcellulose, polydextrose, polyethtylene oxide, povidone, sodium alginate, starch paste, pregelatinized starch, sucrose, tragacanth, low-substituted hydroxypropyl cellulose, glucose, sorbitol.

A suitable filler may be selected from, for example, starch derivatives, such as corn starch, potato starch or rice starch, polysaccharides such as dextrins, maltodextrins, dextrates, microcrystalline cellulose, powdered cellulose, mixture of microcrystalline cellulose and guar gum, coprocessed blends of microcrystalline cellulose; and polyhydric alcohols, such as xylitol and sorbitol.

A disintegrant may be selected from, for example, alginic acid, carbon dioxide, carboxymethylcellulose calcium, carboxymethylcellulose sodium, microcrystalline cellulose, powdered cellulose, croscarmelose sodium, crospovidone, sodium docusate, gaur gum, hydroxypropyl cellulose, methylcellulose, polacrilin potassium, poloxamer, povidone, sodium alginate, sodium glycine carbonate, sodium lauryl sulfate, sodium starch glycolate, starch, pregelatinized starch, low-substituted hydroxypropyl cellulose.

A glidant may be selected from, for example, calcium silicate, powdered cellulose, starch, talc, colloidal silicon dioxide.

A lubricant may be selected from, for example, magnesium stearate, stearic acid, sodium stearyl fumarate, magnesium lauryl sulphate, talc, polyethylene glycol, and glyceryl behenate.

A suitable essential oil may be selected from Bergamot oil (extracted from Citrus aurantium L. subsp. bergamia Wright et Arn.); Ylang ylang oil (extracted from Cananga odorata Hook. f and Thoms.); Jasmine essential oil (extracted from Jasminum officinale L.). In one embodiment, a mixture of essential oils comprises equal portions totaling about 0.01% to about 1% w/w, preferably about 0.1% w/w of the total composition. Other essential oils are possible.

A suitable sweetener may be selected from sugars such as sucrose, lactose and glucose; cyclamate and salts thereof; saccharin and salts thereof; and aspartame.

A flavouring agent may be selected from natural or synthetic flavours such as, for example, strawberry flavour, wild cherry flavour, green apple flavour, spearmint flavour and peppermint flavour.

In various embodiments, one or more of the ingredients of the composition is an organic ingredient.

In a further optional embodiment of the invention, the sustained release oral composition further comprises a coating. The coating material may be selected from materials known to a person skilled in the art.

Further disclosed herein is a combined immediate release and sustained release oral formulation of cannabinoid wherein the dissolution profile of the oral composition releases 5% to 20% within 20 min to 1 hour and greater than 80% within more than 6-10 hours of the cannabinoid content of the formulation.

The preferred cannabis formulation dosage-unit of the present invention contains two fractions of oil-based compounds. A liquid and transparent fraction, which contains pure cannabinoid extract dissolved in organic coconut. This fraction is responsible for the quick onset of the therapeutic effects within 20-60 minutes. A consolidated Cannabinoid-Lipid-based drug delivery systems (LBDDS) fraction, which is responsible for a gradual and long lasting therapeutic effect (6-8 hr) due to a proposed constant and steady release of active cannabinoids.

The formulation contains a pure extract of cannabinoids, monoglyceride and diglyceride (E471), combined with carrageenan which is known for its controlled release properties and organic coconut oil.

The compositions disclosed herein are beneficial in treating and/or reducing the symptoms of a variety of diseases and disorders responsive to treatment with cannabis, preferably cancer related cachexia and anorexia syndrome, but including but not limited to pain associated with cancer and side effects of chemotherapy including nausea. Although the cannabis extract disclosed herein can be formulated for different modes of administration the preferred formulation is an oral formulation for immediate release, sustained release or a combination of immediate release and sustained release.

Persons skilled in the art are aware of the best modes of administration for cannabinoids.

The useful dosage to be administered and the particular mode of administration will vary depending upon such factors as the age, weight of the particular subject, the therapeutic or diagnostic use contemplated, and the form of the formulation.

The “therapeutically effective dose” as used herein is thus determined by such considerations as are known in the art. The dose must be effective to achieve improvement in at least one parameter comprising weight gain of at least 10% from baseline weight, improvement in appetite, improvement in caloric intake, reduction in TNF-alpha level, analgesic effects, antitumor activity, cancer cells cytotoxic effect, antidepressant, an anxiolytic, neuroprotective, anti-psychotic improved quality of life or QoL assessment (e.g. as measured by EORTC C30 and Anorexia/Cachexia Therapy assessment by FAACT questionnaire), improved muscle mass and/or muscle strength and any combination thereof.

Provided herein are compositions and methods for treating cannabinoid responsive diseases and disorders, preferably CACS, by administering an effective amount of a sustained release or combined immediate release and sustained release composition to a subject in need thereof. An effective amount is an amount sufficient to eliminate or to alleviate symptoms. “Sustained release” means that the active cannabinoids are released from the composition over time so that their plasma concentration is maintained within the therapeutic window (above the therapeutically minimal effective concentration but below toxic levels) over an extended period of time of more than 4 hours, preferably between 4houre and 8 hours. Pilot studies in the clinic of the formulations described herein have surprisingly found that the therapeutic effect of a 5 mg combined immediate and sustained release dose lasts up to about 8 hours and the onset of the therapeutic effect is from 20-60 min. The dosage form is preferably administered once per day, for example in the morning, with an optional additional administration in the afternoon, evening or night to achieve a complete 24 hour coverage of the beneficial therapeutic effects.

Yet another embodiment of the present invention is related to the process for the preparation of immediate and sustained release oral formulation of cannabinoid extract. The method for the preparation of a combination sustained release (SR) and immediate release (IR) oral composition of a cannabinoid includes the steps of: (a) mixing a cannabinoid extract with an edible oil to formulate an immediate release (IR) formulation; (b) filling a capsule with said IR formulation; (c) freezing said capsule of step b at −20° C.; (d) mixing a cannabinoid extract with at least one LBDDS agent and optionally an edible oil to formulate a slow release (SR) formulation; (e) thawing said freezed capsule of step c; and (f) adding said SR formulation to said capsule of step e.

In some embodiments, a sustained oral composition of a cannabinoid is preferred and the process for preparing such sustained release oral composition of a cannabinoid includes steps of mixing a cannabinoid extract with at least one LBDDS agent and optionally an edible oil to formulate a slow release (SR) formulation; and filling a capsule with said SR.

In some embodiments of the methods, the method further includes the step of milling, drying, compressing or filling capsules, preferably filling gelatin capsules.

The dosage form of the present invention may be prepared using conventional techniques employed in the art for mixing, compaction, granulation, milling, drying, compressing and/or filling in capsules. The oral formulation may be selected from sprinkle granules or powder for reconstitution in a suspension, tablet, soluble tablet, rapidly disintegrating tablet, orally disintegrating tablet, rapidly disintegrating film, orally disintegrating powder for capsules, suspension or sachets, effervescent tablet, a chewable tablet, water dispersible tablet, orodispersible tablet, a chewing gum and suspension.

The individual dosage forms may be packaged together in the form of a kit for treating a disease symptom or syndrome, preferably CACS, optionally with instructions for use. Alternatively, the individual dosage forms may be individually packaged, as in foil envelopes or in a blister pack.

The aspects and embodiments provided herein have been described in an illustrative manner, and it is to be understood that the terminology used is intended to be in the nature of words of description rather than of limitation.

Many modifications and variations are possible in light of the above teachings. It is, therefore, to be understood that within the scope of the appended claims, the invention can be practiced otherwise than as specifically described.

The present disclosure is illustrated in detail below with reference to examples, but is not to be construed as being limited thereto.

Citation of any document herein is not intended as an admission that such document is pertinent prior art, or considered material to the patentability of any claim of the present disclosure. Any statement as to content or a date of any document is based on the information available to applicant at the time of filing and does not constitute an admission as to the correctness of such a statement.

### EXAMPLES

Without further elaboration, it is believed that one skilled in the art can, using the preceding description, utilize the present invention to its fullest extent. The following preferred specific embodiments are, therefore, to be construed as merely illustrative, and not limitative of the claimed invention in any way.

### Example 1

### Cannabis Capsules Formulation

Lipid-based formulations for oral delivery of lipophilic drugs:

There are various techniques to formulate lipophilic drugs and improve bioavailability.

Lipid-based formulations were shown to improve the biopharmaceutical performance of lipophilic drugs compared to a conventional dosage form. There is typically an increase of oral bioavailability, but other effects like better linearity of exposure or less variability within and between subjects may be observed as well. Lipid-based drug delivery systems (LBDDS) are a key technology to formulate lipophilic compounds. Lipid-based excipients demonstrate several mechanisms by which drug absorption is promoted.

Monoglyceride is a molecule with one glycerol moiety covalently bonded to a fatty acid chain via an ester bond. Diglyceride is a molecule with one glycerol moiety covalently bonded to two fatty acid chains via ester bonds. The mixture of monoglyceride and diglyceride (Glice-E471) act as a type I LBDDS.

Formulation:

The cannabis capsules of the present invention contain two fractions of oil-based compounds. A liquid and transparent fraction, which contains pure cannabinoid extract dissolved in organic coconut. This fraction is responsible for the quick onset of the therapeutic effects within 20-60 minutes. A consolidated Cannabinoid-LBDDS fraction, which is responsible for a gradual and long lasting therapeutic effect (6-8 hr) due to a proposed constant and steady release of active cannabinoids. The formulation contains a pure extract of cannabinoids, monoglyceride and diglyceride (E471), combined with carrageenan which is known for its controlled release properties and organic coconut oil.

Cannabinoid Concentrations:

The two highly abundant cannabinoids in cultivated cannabis plants are THC and CBD.

Through cultivation, most of the medical cannabis plants contain high concentration of THC and low concentration of CBD and other cannabinoids.

The capsules of the present invention contain at least one of the following ratios and amounts of active ingredients:

1. 10 mg of active cannabinoids from which THC is 9.5 mg and CBD is 0.5 mg

2. 5 mg of active cannabinoids from which THC is 4.75 mg and CBD is 0.25 mg

Physical, Chemical, and Pharmaceutical Properties and Formulation:

TABLE 1
Cannabis Capsules

| Name | Cannabis Capsule |
|---|---|
| Dosage form | 5 mg/10 mg |
| Manufacturer | Seach Ltd. |
| Description | Oil Capsule |
| Route of administration | Oral |

The Drug Substance (DS) is Cannabis oil extract.

The Drug Product (DP) contains Coconut oil, Carrageenan Lota/Kappa (E407), Mono and Diglyceride (E471).

The composition of 1 capsule of 5 mg is described in the following table:

TABLE 2
Composition of 5 mg cannabis capsules

| | | Relative | |
|---|---|---|---|
| Material | Amount | amount (V/V) | Manufacturer |
| Cannabis | 5 mg | 0.010 | Seach Ltd |
| extract | | | |
| Carrageenan | 2.5 mg | 0.005 | TBK |
| Lota/Kappa | | | |
| Mono and | 37.5 mg | 0.075 | ® Palsgaard |
| Diglyceride | | | |
| Coconut oil | 453 ul | 0.920 | Maryal |

Appearance: Cannabis Capsules are vegetable based, white capsules.

Storage: Store at 4° C.

### EXAMPLE 2

### Manufacturing of the Cannabis Capsules

The cannabis capsules are produced by a cannabis approved grower and manufacturer. The Cannabinoid profile was tested in Izun Pharma a licensed for laboratory tests on cannabis products. The cannabis Pharmaceuticals' personnel will produce the capsules and GMP rules will be followed and inspected.

Manufacturing Process Overview:

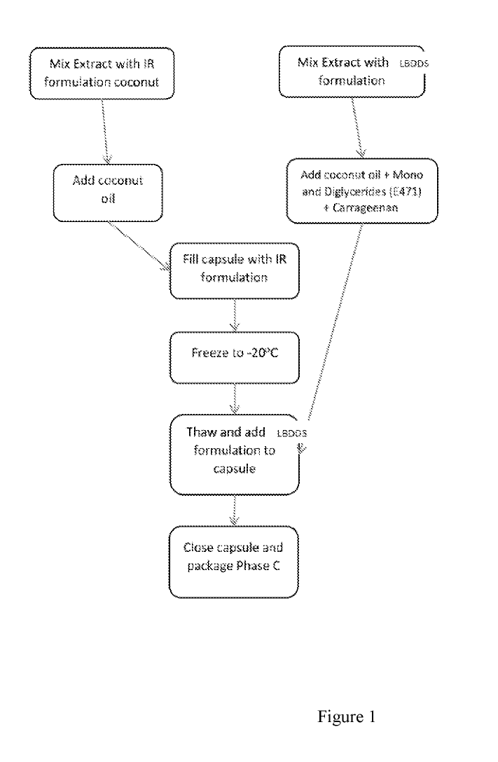

Reference is now made to FIG. 1, schematically presenting embodiments of the manufacturing process of the cannabis capsules of the present invention. The manufacturing process involves mixing the active extracts with IR formulation and capsules formulation. The capsules are filled with the two formulations in two phases. The IR formulation is filled in the capsule and then freeze to −20C. The capsules formulation creates a gel like solution after being poured to the capsule. The LBDDS mixture formulation is added to the capsule containing the IR formulation mixture. The capsule, which now contains two phases, is closed and packaged.

Quality Tests for Determination of the Cannabis Capsules Characteristics

The In Process Controls (IPC) and the Product Release Criteria

The capsules manufacturing process is tightly controlled and all measures are taken to keep the highest level of quality. The samples are stringently tracked throughout the manufacturing process, as described in Table 3.

TABLE 3
IPC's and release criteria of the cannabis capsules

| | | Acceptance | |
|---|---|---|---|
| Manufacturing stage | Test | Criteria | Comments |
| Phase A | Weight | 286 mg ± 10% | IPC |
| Phase B | Weight | 517 mg ± 10% | IPC |
| Phase C | Weight | 557 mg ± 10% | Release |

Cannabis Capsules 5 mg/10 mg Final Characterization:

Identity test for Active materials concentration within the extract solution—CBD/TCH/CBN. Test is used also to detect Nitrate residues.

Final microbiology test will be performed to the filled capsules.

Regulation:

The capsules are authorized under the definition of a cannabis oil product.

### Example 3

### Use of the Cannabis Capsules of the Present Invention as Treatment to Improve Cancer Related Cachexia and Anorexia Syndrome in Advanced Cancer Patients, Pilot Study.

Purpose of the Study:

The main purpose in the treatment of patients with advanced cancer and CACS is to prolong life and to improve quality of life (QoL) as far as possible. QoL in patients with CACS is directly related to loss of appetite and loss of weight. Cannabis pills are given in Israel to advanced cancer patients with various symptoms in order to improve their QoL. There is data on safety/toxicity of cannabis, and these pills are given under the regulations of the Israel Ministry of Health.

The purpose of this study is to examine the influence of the cannabis capsules of the present invention on improving loss of appetite and loss of weight.

Intended Use and Indications:

The treatment of patients with advanced cancer and CACS.

Study Design:

Open controlled trial, pilot study.

Study Population:

Patients with advanced cancer and CACS (loss of appetite and loss of weight).

Eligibility Criteria:

1. Age above 18 years

2. Histological evidence of an incurable malignancy

3. Estimated life expectancy ≥3 months

4. Performance status ≤2 (ECOG classification)

5. Self-report of weight loss of at least 3 kg during the preceding 2 months and/or a dietitian-estimated caloric intake of less than 20 calories/kg of body weight per day

6. Patient believes that loss of appetite or loss of weight is an ongoing problem for him

7. Use of chemotherapy or radiotherapy is permitted

8. Sign of written informed consent

No. of Subjects:

Step 1: 25 patients

Step 2: Additional 15 patients to maximum of 40 patients

Study Duration:

Three months

Data Analysis:

Data analysis is carried out by the statistical software SPSS 18.

A p-value of <0.05 suggests statistical significance for all outcome measures.

Study Endpoints:

The primary objective of the study is weight gain of ≥10% from baseline weight. The secondary end-points include: improvement in appetite, improvement in caloric intake, and reduction in TNF-alpha level.

Study Procedures:

Patients are treated initially for 3-4 days with 1×10 mg capsules per day or 1×5 mg capsules per day for gradual adaptation. From the 5th day, patients are treated with 2×10 mg capsules per 24 hours, or with 2×5 mg capsules per 24 hours, respectively.

Duration of Follow up:

Six Months

### LIST OF ABBREVIATIONS

AE Adverse Event

EC Ethics Committee

ICF Informed Consent Form

IFU Instructions for Use

IRB Institutional Review Board

SAE Serious Adverse Event

CRF Case Record Form

SOP Standard Operation Procedure

IR Immediate Release

THC Tetrahydrocannabinol

CBD Cannabidiol

Study Rationale

1. The main purpose in the treatment of patients with advanced cancer and CACS is to prolong life and to improve quality of life (QoL) as far as possible.

2. QoL in patients with CACS is directly related to loss of appetite and loss of weight.

3. Cannabis pills are given in Israel to advanced cancer patients with various symptoms in order to improve their QoL.

4. There is data on safety/toxicity of cannabis, and these pills are given under the regulations of the Israel Ministry of Health.

5. The influence of the pills on improving loss of appetite and loss of weight is not known.

6. The possible mechanism for such an influence has never been tested.

Study Design & Objective

Overview

Single-center, open controlled trail with patients with advanced cancer and CACS (loss of appetite and loss of weight)

Study Objective

Primary Objective:

Weight gain of ≥10% baseline weight

Secondary Objective:

1. Improvement in appetite

2. Improvement in caloric intake

3. Reduction in TNF-alpha level

4. Correlation between THC levels and primary outcome.

5. Safety profile of cannabis pills

Study Duration

Study Period

The focus of the study is the change of body weight and caloric intake during the first three months of the study. According to patient will, the study medication (Cannabis capsules) is given free of charge for another three months, for total period of six months, with minimal evaluation every six weeks.

Cannabis Treatment

The Following Data Was Obtained From Patients Treated With the Capsules Through Questioners:

The preferable treatment with Cannabis capsules of the present invention is 2×10 mg capsules per 24 hr, or 2×5 mg capsules per 24 hr. First intake is preferable in the morning. Onset of the effect is from 20-60 minutes and the beneficial effect is observed for 4-8 hours. After 2-5 hours, eating increases the effect. The second dosage could be administered after 8 hours according to patient's need or before sleep for patients who suffer from sleep deprivation.

Dose Reductions:

In this study, patients are treated initially for 3-4 days with 1×10 mg capsules per day, or with 1×5 mg capsules per day for gradual adaptation. From the 5th day, patients are treated with 2×10 mg capsules per 24 hours, or with 2×5 mg capsules per 24 hours, respectively.

However, since some patients may suffer from side effects mainly, dizziness and or anxiety, dosage for these patients is reduced to 10 mg per day or 5 mg per day.

Reducing Side Effects:

Recommended therapeutic dosages of the cannabis capsules range between 5 mg to 20 mg per day, depending on indication, individual response and tolerance.

The treatment begins with doses of 10 mg or lower, preferably 5 mg.

Patients accommodate gradually to the effects and learn to how to incorporate the effect with daily routine.

Patient supervision: In the first ten days of treatment patients should be accompanied by a caregiver, friend or a relative that is familiar with possible side effects and their treatment.

High doses can produce either mild anxiety dizziness due to a decrease in blood pressure. It is advisable to sit comfortably and drink natural citrus fruit juice. In case of a sense of dissociation, “disconnected”, a shower can significantly ease.

It is always advisable to be in a ventilated area and natural as possible. Closed and crowded places can become a distress. Safe social environment allows the patient to focus on the healing aspect of the therapy.

Contraindications and Precautions

Contraindications

1. Abnormal sensitivity to individual components of the preparations.

2. Severe personality disorders and psychoses

3. Strict precautions in:

4. Pregnant and breast-feeding women.

5. Severe cardiovascular diseases

6. Hepatitis C

7. Addictive disorders

Precautions

1. Keep away from children.

2. During a course of cannabinoids the patient's ability to drive vehicles and operate machinery safely may be impaired.

Study Population

Step 1: 25 patients

Step 2: Additional 15 patients to maximum of 40 patients

Subject Selection Criteria

Subject Inclusion Criteria

1. Age above 18 years

2. Histological evidence of an incurable malignancy

3. Estimated life expectancy ≥3 months

4. Performance status ≤2 (ECOG classification)

5. Self-report of weight loss of at least 3 kg during the preceding 2 months and/or a dietitianestimated caloric intake of less than 20 calories/kg of body weight per day

6. Patient believes that loss of appetite or loss of weight is an ongoing problem

7. Use of chemotherapy or radiotherapy is permitted

8. Sign of written informed consent

Subject Exclusion Criteria:

1. Ongoing use of tube feedings or parental nutrition

2. Edema or ascites

3. Central nervous system metastases or brain tumors (patients with stable disease in the brain 28 days after treatment can be included in the study)

4. Treatment with adrenal corticosteroids (except for short-term dexamethasone during time of chemotherapy), androgens, progestational agents or other appetite stimulants within the previous two weeks

5. Insulin-requiring diabetes

6. Pregnancy or lactation or unwillingness to use oral contraceptives

7. Other life-threatening medical conditions

8. Anticipated alcohol or barbiturate use during the study period

9. Mechanical obstruction of the alimentary tract, malabsorption, or intractable vomiting

10. Use of cannabis or synthetic cannabinoids in the last four weeks

Study Visits and Procedures

Assessment:

Base-Line Assessment:

1. Physician anamnesis and physical examination

2. Complete blood cell count (CBC) and biochemistry test: electrolytes, renal and liver function tests, albumin level, total cholesterol level

3. Blood test for TNF-alpha level

4. Nutrition evaluation, including daily caloric calculation (by using 3 day food diary) and weighting the patient

5. QoL assessment by EORTC C30 and Anorexia/Cachexia Therapy assessment by FAACT questionnaire

6. Evaluation of muscle strength by using hand dynamometer as estimation for muscle mass. Treatment assessment:

1. Physician anamnesis including toxicity assessment according to CTCAE recommendations for acute toxicity, and physical examination including weighing the patient every two weeks in the first month, every month in the coming two months, and every six weeks in the next three months

2. CBC, biochemistry blood test, TNF-alpha level on day 1, and after three months

3. Nutritional intake evaluation on the first week and after three months, based on daily caloric calculation of three day food diary

4. Muscle strength evaluation on day 1 and after three months

5. QOL assessment by EORTC C30 and Anorexia/Cachexia Therapy assessment by FAACT questionnaire on day 1 and after three and six months

6. Safety assessment for early psychiatric side-effects by the Community Assessment of Psychic Experiences (CAPE) questionnaire on day 1 and after 2 weeks and after 3 months.

7. Urine THC-levels one day 1, 2 weeks, 3 months.

Quality-of-Life (QOL) Assessment

QOL is assessed using the European Organization of Research and Treatment of Cancer core questions on the Quality of Life Questionnaire, version 2 (QLQ-C30) and the Anorexia/Cachexia Therapy (FAACT) questionnaire.

Nutrition Assessment

1. Daily caloric calculation is based on the three day food diary. On day 1, the patient will meet the dietitian and receive guidance and instruction of how to complete the diary. The cannabis pills are given on day 4, after bringing back the three day diary. After three months, the patient will again do a three day food diary and caloric calculation will be made.

2. The caloric calculation will be made with the Ministry of Health Computer software “Zameret”.

3. The patient will have telephone support from the dietitian as needed during the days of the diary completion.

4. The cannabis capsules will be given on day 4, after bringing back the three day diary.

Safety Assessment:

Community Assessment of Psychic Experiences (CAPE) questionnaire to evaluated possible early psychiatric side-effects will be given in 3 time points, together with measuring the THC level in the urine. Evaluation of other side-effects will be done in every physician visit.

TABLE 4
Visits and follow up Table

| | Visit No. | | | | | | |
|---|---|---|---|---|---|---|---|
| | 1 | 2 | 3 | 4 | 5 | 6 | 7 |
| | (Day 1) | (2 W) | (1 M) | (2 M) | (3 M) | (4.5 M) | (6 M-end) |
| Physician anamnesis | X | X | X | X | X | X | X |
| Toxicity assessment | | X | X | X | X | X | X |
| Physical examination | X | X | X | X | X | X | X |
| Patient weight | X | | X | X | X | X | X |
| QOL assessment (EORTC-C30) | X | | | | X | | X |
| FAACT questionnaire | X | | | | X | | X |
| CAPE questionnaire | X | X | | | X | | |
| CBC | X | | | | X | | |
| Biochemistry test | X | | | | X | | |
| TNF-alpha level | X | | X | | | | |
| Urine THC level | X | X | | | X | | |
| Nutrition evaluation | X | | X | | | | |

Subject Identification and Confidentiality

An eligible subject's case—subject identification code—will be composed of a three-digit number that represents the sequential serial number of study screening at the site as well as two-letters subject initials (such as 001—AB).

All reports and communications relating to study subjects will identify the subject only by this number and initials. The subject identification will be kept confidential at the investigational site.

A dedicated “subject identification log”, which includes the subject's identifying details (e.g. name, ID number, telephone number) together with his/her identification number, will be kept in the investigational site. This log will be kept only in the investigational site and will not be transferred to the Sponsor.

Procedure relevant and required forms will be completed.

Subject Withdrawal or Discontinuation

Each subject will be informed of his right to withdraw from the study at any time and for any reason.

The subject's participation in this study may be discontinued due to (but not limited to) the following reasons:

1. Request of regulatory agency, or Sponsor or primary care physician or Investigator

2. The subject withdrew consent.

3. An adverse event, which jeopardize further subject's participation in study

4. The subject is unwilling or unable to continue the study or is lost-to-follow-up

5. The subject is non-compliant with study procedures/study protocol

6. The investigator decides that withdrawal from the study is in the best interest of the subject.

7. The subject requires medications, which in the opinion of the investigator may interfere with continuation of the study.

8. Any clinically significant change in subject's medical condition.

The reasons for any subject withdrawal will be recorded on the study completion form of the CRF. The Investigator will inform the Sponsor in writing of the subject's early withdrawal for any reason.

Upon withdrawal from the study, any time after Visit 2 has taken place, the subject will return to the clinic and all termination visit procedures will be performed.

If withdrawal is caused by an adverse event that the investigator considers may be related to the study procedures, it will be reported to the IRB and to the Sponsor.

Statistics Methods

The primary objective of the study is weight gain of ≥10% from baseline weight. The secondary end-points include: improvement in appetite, improvement in caloric intake, and reduction in TNF-alpha level.

To test the null hypothesis (H0) that cannabis pills can improve body weight by more than 10%, the number needed to treat was calculated according to true response probability of less than 5%.

This calculation with the same primary end-point that achieved 3% true response on dronabinol and 11% on megastrol was based on the results of a Phase III study (31).

Based on a significance level of 0.05 (alfa) and a power of 0.90, the sample size in the first stage should be 21 patients. If only one patient achieves the primary end-point, the study will be terminated.

Bivariable logistic regression analysis will be using for the calculation of the odds ratios (OR) with 95% confidence intervals (CI) and p values in bivariable analysis to identify an association between patient characteristics and response rate. Multivariable Logistic Regression analysis will be performed to assess patient characteristics with a higher response probability. Variables were selected as candidates for the multivariate analysis on the basis of the level of significance of the bivariable association with response rate (p<0.1).

All outcome measures of appetite rate (FAACT questionnaire), caloric intake, TNF-alpha level, and QQL will be calculated based on published normative data. Median scores will be calculated for each outcome measure as the difference between baselines (T0), end of the intensive evaluation period in the first three months (T3), and in the end of the additional three months of treatment (T6). Differences between T0 and T3 will be tested using the Wilcoxon paired nonparametric test.

Data analysis is carried out by the statistical software SPSS 18. A p-value of <0.05 suggests statistical significance for all outcome measures.

Data Management

Data Collection

Data from each subject is recorded in source data and transmitted to case report forms (CRFs). The data is inserted manually and quality check for errors and omissions is performed to ensure the accuracy of the entered data.

The investigator will retain originals of CRFs, subject consent forms, and study data as permanent records for a period of 15 years or until the data is no longer required for regulatory purposes (the longest between these two).

The CRFs should be reviewed by the sponsor's appointed monitor for accuracy and completion (signatures, dates, adverse events, serious adverse events, protocol deviations).

Monitoring Plan

Monitoring functions shall be performed in compliance with Good Clinical Practices, EN ISO 14155:2011, as outlined in 21CFR § 821.43(d) and 21CFR § 812.46, and according to any other local regulations.

The Sponsor will appoint a Clinical Monitor for this study. The Clinical Monitor should be qualified by training and experience to oversee the conduct of the study. The Clinical Monitor's responsibilities include maintaining regular contact with the investigational site, through telephone contact and on-site visits, to ensure that: 1) the study protocol is followed; 2) that complete, timely, and accurate data are gathered; 3) that problems with inconsistent and incomplete data are addressed; and 4) that complications and Unanticipated Adverse Events are reported to the Sponsor.

Deviations From Study Plan

The Investigator should document and explain any deviation from the approved protocol and file waivers received from the sponsor, if applicable. The documented deviation should be submitted to:

The Sponsor

The IRB

Any subject treated in a manner that deviates from the protocol, or who is admitted into the study but is not eligible according to the protocol, may be ineligible for analysis and thereby compromises the study.

This study will be conducted in compliance with the protocol after approval of the local Ethic Committee, in accordance with the ethical principles that have their origin in the Declaration of Helsinki and in compliance with Good Clinical Practice (GCP).

No deviation from the protocol, after sponsor's and Ethic Committee approval will be implemented without the prior review and approval except where it may be necessary to eliminate an immediate hazard to the subject. In such case, the significant safety deviation will be reported to the IRB and the sponsor as soon as possible.

A copy of the Clinical protocol, Informed Consent Form (ICF), Investigator Brochure (IB) and any relevant material (as required by IRB) must be submitted to the IRB. Written approval of the protocol, the Informed Consent Form and advertising material must be obtained prior to initiation of the study.

Investigational Product (IP) Accountability

The Sponsor will provide the study site with the IP prior to the start of the study. The IP will be marked “for investigational use only” and the investigator is responsible for storing them in a secure place to avoid unauthorized use.

Immediately upon completion of the study of each individual all unused IP (if remains) will be returned to Sponsor. Relevant accountability forms will be managed.

Regulatory and Ethical Compliance

This clinical study was designed and shall be implemented and reported in accordance with the ICH Harmonized Tripartite Guidelines for Good Clinical Practice, with applicable local regulations and with the ethical principles of Declaration of Helsinki.

Amendments to the Protocol

Protocol modifications (Amendments) must be confirmed in writing by the Sponsor prior to implementation.

All major protocol amendments must be approved by the IRB prior to implementation. No protocol amendments should be adopted without prior written approval from the IRB except in the following cases:

In order to eliminate immediate hazard to the subjects

Changes involving only logistical or administrative aspects of the trial.

Protocol Adherence

Investigators ascertain they will apply due diligence to avoid protocol deviations. If the investigator feels a protocol deviation would improve the conduct of the study, this must be considered a protocol amendment, and unless such an amendment is agreed upon by sponsor and approved by the IRB it cannot be implemented. All significant protocol deviations will be recorded and reported.

Informed Consent Process

The Investigator or his designee in accordance with institutional policy will obtain an Informed Consent, acceptable by the institution's Ethics Committee. A written consent form bearing the full name and signature of the subject will be obtained from each subject. The signed Informed Consent constitutes a confidential document and therefore should be archived in the Investigator File or in the Site's Record.

The written Informed Consent form and any other written information to be provided to subjects should be revised if any important new information becomes available that may be relevant to the subject's consent. The Ethic Committee must approve any revised written Informed Consent form and written information before it is made available for subject signature. The subject should be informed in a timely manner if new information becomes available that might be relevant to the subject's willingness to participate in the study. The communication of this information should be documented.

Neither the investigator, nor the trial staff, should coerce or unduly influence a subject to participate or to continue to participate in a trial.

The investigator, or a person designated by the investigator, should fully inform the subject of all pertinent aspects of the study including the approved written informed consent.

Before informed consent may be obtained, the investigator, or a person designated by the investigator, should provide the subject ample time and opportunity to inquire about details of the study and to decide whether or not to participate in the study. All questions about the study should be answered to the satisfaction of the subject.

Prior to a subject's enrollment into the study, the approved informed consent form should be personally signed and dated by the subject and by the person who is authorized to conduct the informed consent discussion.

Responsibilities of the Investigator and IRB/IEC

The protocol and the proposed informed consent form must be reviewed and approved by a properly constituted Institutional Ethics Board (IRB) before study start. A signed and dated statement that the protocol and informed consent have been approved by the IRB must be given to sponsor before study initiation. Prior to study start, the investigator is required to sign a protocol signature page confirming his/her agreement to conduct the study in accordance with these documents and all of the instructions and procedures found in this protocol and to give access to all relevant data and records to monitors, auditors, Clinical Quality Assurance representatives, designated agents of sponsor, IRBs, and regulatory authorities as required. If an inspection of the clinical site is requested by a regulatory authority, the investigator must inform sponsor immediately that this request has been made.

Risks and Benefits of the Investigational Product and Study

Anticipated Clinical Benefits

It is expected that patients will benefit from one or more of the following: Increased appetite, nausea reduction, pain relief, increased motivation and activity, increased attention, improved sleep.

Anticipated Serious Adverse Events

No Serious AEs are Expected.

Precaution to Minimize Risk

To minimize the risks involved in the study, all actions will be taken by trained professionals with expertise in the medical field.

The cannabis capsules of the present invention are produced in a cannabis approved facility-Izun Pharma. The site is certified for ISO 9001:2008 and ISO 13485 for medical device. Approved personnel will produce the capsules and GMP rules will be followed and inspected.

The following Adverse Events may occur because of the use of the cannabis capsules of the present invention:

1. Dizziness

2. Anxiety

3. Short-term memory loss.

4. Nausea

5. Dry mouth

6. Sweating

7. Dissociation

In case of AE and SAE, subjects will be treated immediately as per routine hospital procedure.

The action taken to treat the adverse event should be recorded on the Adverse Event CRF.

Serious Adverse Events Reporting

Serious Adverse events (SAE) occurring in subjects enrolled in the study must be evaluated and reported to the sponsor within 24 hours from acknowledgment by site and site personnel. SAE will be reported the local IRB and also to the ministry of health in Israel (MOH) as per local guidance.

Definitions of Adverse Events

An adverse event (AE) is any untoward medical occurrence (sign, symptom, illness, abnormal laboratory value, or other medical event) in a subject. This definition does not imply that there is a relationship between the adverse event and the device under investigation.

A serious adverse event (SAE) is any adverse event that:

Led to death;

Led to a serious deterioration in the health of the subject that;

Resulted in a life-threatening illness or injury;

Resulted in a permanent impairment of a body structure or a body function;

Required in-patient hospitalization or prolongation of existing hospitalization;

resulted in medical or surgical intervention to prevent permanent impairment to body structure or a body function.

Led to Fatal Distress, Fatal Death or a Congenital Abnormality or Birth Defect

An Unanticipated Adverse Device Effect—any serious adverse effect on health or safety or any life-threatening problem or death caused by, or associated with, a device, if that effect, problem, or death was not previously identified in nature, severity, or degree of incidence in the investigational plan or application, or any other unanticipated serious problem associated with a device that relates to the rights, safety, or welfare of subjects.

All adverse events will be graded as follows:

| | |
|---|---|
| Mild: | Sign or symptom, usually transient, requiring no special |
| | treatment and generally not interfering with usual activities. |
| Moderate: | Sign or symptom, which may be ameliorated by simple |
| | therapeutic measures; yet, may interfere with usual activity. |
| Severe: | Sign or symptom that are intense or debilitating and that |
| | interfere with usual activities. Recovery is usually aided by |
| | therapeutic measures. |

The relationship of the adverse event to the treatments or procedures is defined as follows:

| | |
|---|---|
| Most probably | Follows a reasonable temporal sequence from study |
| related: | treatment, and cannot be reasonably explained by |
| | known characteristics of the patient's clinical data |
| | or the procedure applied. |
| Possible | Follows a reasonable temporal sequence from study |
| related: | treatment but could have been produced by the |
| | patient's clinical state or by the procedures |
| | regardless of the treatment |
| Probably not | Temporal association is such that the treatment is not |
| related: | likely to have had any reasonable association with the |
| | observed event. |
| Not related: | No relationship to treatment activation is perceived. |

Adverse Event Reporting

All adverse events will be recorded on the adverse events page of the CRF. Severity and relationship to study procedure and investigational product will be assigned by the investigator as described in the section above.

Adverse events will be recorded after the subject has been admitted to the Visit 1 and throughout the study including the follow-up termination visit. Adverse events should be reviewed and updated at each subsequent visit and during any phone contact with the subject.

Any SAE, whether deemed study procedure/investigational product-related or not, must be reported to the site Ethics Committee and to the sponsor by email with confirmation of receipt mode and by telephone and by fax, as soon as possible after the investigator has become aware of its occurrence even if not all the information is available at the time of initial contact:

The investigator must complete a SAE Form, and send it, via fax, to the Sponsor within 24 hours of becoming aware of the event. Accompanying documentation, such as copies of hospital case reports, autopsy report, and other documents when applicable, should be sent as soon as they are available.

The site's Ethics Committee must also be duly notified and dealt with, according to the Hospital and Ministry of Health regulations.

Vulnerable Population

No vulnerable population will be included in this study

Suspension or Premature Termination of the Study

The Sponsor reserves the right to discontinue the study at any time for any reason based on (but not exclusively) the following criteria:

1. Technical problems in the study procedures.

2. Unexpected adverse effects.

3. The Principal Investigator's or the Ethics Committee recommendation

4. Poor performance or compliance of the clinical site

5. Company considerations

In case of premature termination of the study, the Ethics Committee will be duly informed according to the local regulations.

### Example 4

### Pharmacological Effect of the Cannabis Formulations of the Present Invention

In one possible embodiment of the present invention the capsules are composed of Cannabidiol (CBD) in an Immediate Release (IR) Formulation and Tetrahydrocannabinol (THC) in a Sustained Release (SR) Formulation. This means that the patient body receptors are exposed first to the CBD and in a later time to the THC.

CBD Mechanism of Action

It is known in the art that CBD can be used as an anticonvulsant and has anti-psychotic effects and may counteract the potential psychotomimetic effects of THC on individuals with latent schizophrenia (https://en.wikipedia.org/wiki/Cannabidiol, incorporated hereinafter as reference). Some reports propose CBD as an alternative treatment for schizophrenia that is safe and well-tolerated. Studies have shown that CBD may reduce schizophrenic symptoms due to its apparent ability to stabilize disrupted or disabled NMDA receptor pathways in the brain, which are shared and sometimes contested by norepinephrine and GABA. Other studies described a double blind, 4 week, explorative controlled clinical trial, compare the effects of purified CBD and the atypical antipsychotic amisulpride on improving the symptoms of schizophrenia in 42 patients with acute paranoid schizophrenia. Both treatments were associated with a significant decrease of psychotic symptoms after 2 and 4 weeks as assessed by Brief Psychiatric Rating Scale and Positive and Negative Syndrome Scale. While there was no statistical difference between the two treatment groups, CBD induced significantly fewer side effects (extrapyramidal symptoms, increase in prolactin, weight gain) when compared to amisulpride. A phase 2 study of 88 schizophrenic patients who failed to respond to a first-line anti-psychotic, had CBD (or placebo) added on. CBD improved positive and cognitive symptoms over six weeks with no severe adverse effects. Studies have shown CBD decreases activity of the limbic system and decreases social isolation induced by THC. CBD has also been shown to reduce anxiety in social anxiety disorder.

The pharmacodynamics of CBD is also known in the art. CBD has a very low affinity for CB₁ and CB₂ receptors but acts as an indirect antagonist of their agonists. While one would assume that this would cause CBD to reduce the effects of THC, it may potentiate THC's effects by increasing CB₁ receptor density or through another CB₁-related mechanism. It may also extend the duration of the effects of THC via inhibition of the cytochrome P-450-3A and 2C enzymes. Recently, it was found to be an antagonist at the putative new cannabinoid receptor, GPR55, a GPCR expressed in the caudate nucleus and putamen. CBD has also been shown to act as a 5-HT_{1A} receptor agonist, an action which may be involved in its antidepressant, anxiolytic, and neuroprotective effects. CBD is an allosteric modulator of μ and δ-opioid receptors. CBD's pharmacological effects have also been attributed to PPAR-γ receptor agonism and intracellular calcium release.

It is suggested that CBD may exert some of its pharmacological action through its inhibition of FAAH, which may in turn increase the levels of endocannabinoids, such as anandamide, produced by the body.

There is some preclinical evidence to suggest that pharmacokinetic interactions of CBD may reduce THC clearance, modestly increasing THC's plasma concentrations resulting in a greater amount of THC available to receptors, increasing the effect of THC in a dose-dependent manner.

THC Mechanism of Action

It is known in the art that THC has mild to moderate analgesic effects, and can be used to treat pain by altering transmitter release on dorsal root ganglion of the spinal cord and in the periaqueductal gray. Other effects include relaxation, alteration of visual, auditory, and olfactory senses, fatigue, and appetite stimulation. THC has marked antiemetic properties. It may acutely reduce aggression (https://en.wikipedia.org/wiki/Tetrahydrocannabinol, incorporated hereinafter as reference).

It is herein acknowledged that due to its partial agonistic activity, THC appears to result in greater downregulation of cannabinoid receptors than endocannabinoids, further limiting its efficacy over other cannabinoids. Without wishing to be bound by theory, it is submitted that while tolerance may limit the maximal effects of certain drugs, it is suggested that tolerance develops irregularly for different effects with greater resistance for primary over side-effects, and may actually serve to enhance the drug's therapeutic window. However, this form of tolerance appears to be irregular throughout mouse brain areas. THC, as well as other cannabinoids that contain a phenol group, possesses mild antioxidant activity sufficient to protect neurons against oxidative stress, such as that produced by glutamate-induced excitotoxicity.

It has been also suggested, from a study in mice, that based on the connection between palatable food and stimulation of dopamine (DA) transmission in the shell of the nucleus accumbens (NAc), THC may not only stimulate taste, but possibly the hedonic (pleasure) value of food as well. The study demonstrates habitual use of THC lessening this heightened pleasure response, indicating a possible similarity in humans. The inconsistency between DA habituation and enduring appetite observed after THC application suggests that cannabis-induced appetite stimulation is not only mediated by enhanced pleasure from palatable food, but through THC stimulation of another appetitive response as well.

THC is been widely researched for its potential medical uses. In April 2014 the American Academy of Neurology published a systematic review of the efficacy and safety of medical marijuana and marijuana-derived products in certain neurological disorders. The review identified 34 studies meeting inclusion criteria, of which 8 were rated as Class I quality. The study found evidence supporting the effectiveness of cannabis extracts and THC in treating certain symptoms of multiple sclerosis.

It is herein suggested that THC helps alleviate symptoms suffered both by AIDS patients, and by cancer patients undergoing chemotherapy, by increasing appetite and decreasing nausea. It is shown to assist some glaucoma patients by reducing pressure within the eye. It is further used in the form of cannabis by multiple sclerosis patients, who use it to alleviate neuropathic pain and spasticity. It is herein further acknowledged that THC also shows antitumor activity in animal studies where it kills cancer cells. Studies in humans have been limited by federal and state laws criminalizing marijuana. In August 2009 a phase IV clinical trial by the Hadassah Medical Center in Jerusalem, Israel started to investigate the effects of THC on post-traumatic stress disorders.

CBD, the second most abundant cannabinoid found in cannabis, is an indirect antagonist against cannabinoid agonists; thus reducing the effects of anandamide and THC agonism on the CB1 and CB2 receptors.

It is further within the scope that the activity of THC result from its partial agonist activity at the cannabinoid receptor CB₁ (K₁=10 nM), located mainly in the central nervous system, and the CB₂ receptor (Kᵢ=24 nM), mainly expressed in cells of the immune system. The psychoactive effects of THC are primarily mediated by its activation of CB₁G-protein coupled receptors, which result in a decrease in the concentration of the second messenger molecule cAMP through inhibition of adenylate cyclase.

Effects of Using CBD in an IR Formulation and THC in a SR Formulation

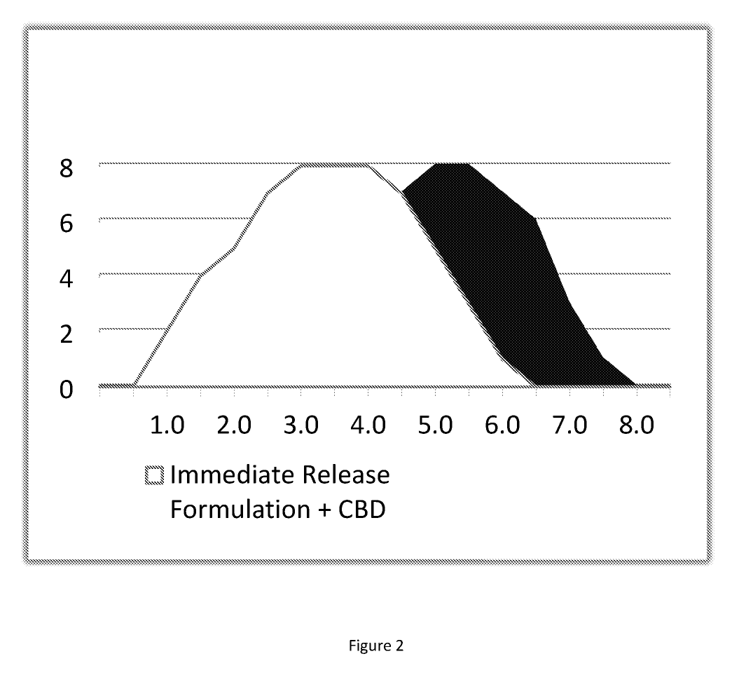

Reference is now made to FIG. 2 showing a schematic representation of the time-dependent release of THC and CBD of the composition of the present invention.

According to one aspect, the capsule of the present invention comprises two fractions. One fraction comprises CDB in an immediate release formulation and a second fraction comprises THC in a sustained or controlled release formulation. As described in FIG. 2, upon administration of the capsule to a patient, first, CBD is introduced with its narcotic effect, and after about 5 hours (e.g. after the patient is asleep) the sedative effect starts and the patient's pain symptoms are significantly reduced.

Thus, according to a further embodiment, using the capsule of the present invention, a time limited psychoactive effect is provided without reducing the active compound potency.

According to a further aspect, the IR formulation delivers THC and then the SR formulation delivers CBD/THCV which is antipsychotic. This type of capsule and protocol enables patients with insomnia to reduce pain (THC) caused by chronic pain and improved their sleep but avoiding the psychotic effect.

It is further within the scope that the formulation of the present invention comprising CBD in a fast release form, increases CB₁ receptor density, inhibits the cytochrome P-450-3A and 2C enzymes, acts as a 5-HT_{1A} receptor agonist, and acts as an anticonvulsant and as an anti-psychotic. Without wishing to be bound by theory, it is submitted that these effects prepare the body for the second phase, which is the release of THC from the cannabis capsule formulation. Some of these effects are mentioned bellow:

The increment of CB1 receptor density potentiate THC's effects, such as analgesic effects, antitumor activity as killer of cancer cells, appetite stimulation and all the other aforementioned effects.

The inhibition of the cytochrome P-450-3A and 2C enzymes by CBD extend the duration of the effects of THC.

The 5-HT1A receptor agonist activity of CBD, results in an effect as an antidepressant, an anxiolytic, and neuroprotective effects.

The anti-psychotic activity of CBD counteracts the potential psychotomimetic effects of THC.

It is therefore within the scope that the cannabis formulation of the present invention is designed to control THC and CBD related beneficial or therapeutic effects in accordance with the appropriate indication or disease treated with the cannabis formulation.

It is further within the scope that the cannabis formulation of the present invention reduces the psychoactive effect of THC without reducing its prolonged therapeutic effects.

It is further within the scope that the cannabis formulation of the present invention affects not only THC and CBD-related effects and receptors, but also the activity and effects of other cannabinoids such as cannabinol (CBN), CBG (Cannabigerol), CBC (Cannabichromene), CBL (Cannabicyclol), CBV (Cannabivarin), THCV (Tetrahydrocannabivarin), CBDV (Cannabidivarin), CBCV (Cannabichromevarin), CBGV (Cannabigerovarin) and CBGM (Cannabigerol Monomethyl Ether).

It is emphasized that this example can be applied to any combination of cannabinoid lipophilic mixtures or extracts.

1.-75. (canceled)

76. An oral composition for the treatment of cancer-related cachexia and anorexia syndrome (CACS), wherein said composition comprising a lipophilic mixture of Cannabidiol (CBD) or derivative thereof in an immediate release formulation and Tetrahydrocannabinol (THC) or derivative thereof in a Lipid-based Drug Delivery System (LBDDS) sustained-release formulation, further wherein the ratio of said THC:CBD is about 95:5% w/w.

77. The composition of claim 76, wherein said immediate release formulation comprises an edible oil and said sustained release formulation comprises at least one of at least one Lipid-based Drug Delivery System (LBDDS) agent or an edible oil.

78. The composition of claim 77, wherein the LBDDS agent is selected from the group consisting of a monoglyceride, a diglyceride, a carrageenan and any mixture thereof.

79. The composition of claim 78, wherein the carrageenan is selected from the group consisting of lambda-carrageenan, kappa-carrageenan, iota-carrageenan and any mixture of the carrageenan.

80. The composition of claim 77, wherein the edible oil in the immediate release formulation and in the sustained release formulation is independently selected from the group consisting of coconut oil, wheat sprout oil, olive oil, sprouted wheat oil, sesame oil, peanut oil, grape seed oil, palm oil, papaya seed oil or any combination thereof.

81. The composition of claim 76, wherein at least one of the following holds true:
a. said composition comprises cannabis extract in a relative amount range of about 0.01 V/V to about 0.02 V/V; or
b. said composition comprises Carrageenan in a relative amount of about 0.005 V/V; or
c. said composition comprises Mono and Diglyceride in a relative amount of about 0.075 V/V; or
d. said composition comprises coconut oil in a relative amount of about 0.92 V/V.

82. The composition of claim 76, further comprising at least one excipient selected from the group consisting of a diluent, a binder, a lubricant, a disintegrant, a flavoring agent, a coloring agent, a stabilizer, a surfactant, a glidant, a plasticizer, a preservative, an essential oil and a sweetener.

83. The composition of claim 76, wherein at least one of the following holds true:
a. the therapeutic effect of said composition has a duration from 4 to 18 hours; or
b. said syndrome is associated with a symptome selected from the group consisting of: weight loss, appetite loss, reduced caloric intake, elevated TNF-alpha level, anorexia, cachexia, reduced Quality-of-Life, mood related conditions, gastrointestinal problems, reduced muscle mass, reduced muscle strength, pain, and any combination thereof.

84. The composition of claim 76, wherein the composition comprising cannabis extract in a relative amount of about 0.01 V/V, Carrageenan in a relative amount of about 0.005 V/V, Mono and Diglyceride in a relative amount of about 0.075 V/V and coconut oil in a relative amount of about 0.92 V/V.

85. The composition of claim 76, wherein the cannabinoids are present in a total amount 2.5 mg to 50 mg per dosage form, preferably 5 mg per dosage form.

86. The composition of claim 76, wherein said composition provides a beneficial effect selected from the group consisting of weight gain of at least 10% from baseline weight, improvement in appetite, improvement in caloric intake, reduction in TNF-alpha level, analgesic effects, antitumor activity, cancer cells cytotoxic effect, antidepressant, an anxiolytic, neuroprotective, anti-psychotic, improvement in quality of life (QoF) and any combination thereof.

87. The composition of claim 86, wherein said improvement in quality of life (QoF) is assessed using the European Organization of Research and Treatment of Cancer core questions on the Quality of Life Questionnaire, version 2 (QLQ-C30) and the Anorexia/Cachexia Therapy (FAACT) questionnaire.

88. The composition of claim 76, wherein the composition is formulated as granules, powder, capsules, gelatin capsule, tablet, film, suspension, sachets, a chewing gum and suspension.

89. A method for the treatment of cancer-related cachexia and anorexia syndrome (CACS) in a subject, comprising steps of:
a. providing an oral composition comprising a lipophilic mixture of Cannabidiol (CBD) compound or derivative thereof—in an immediate release formulation and Tetrahydrocannabinol (THC) or derivative thereof in a Lipid-based Drug Delivery System (LBDDS) sustained-release formulation, further wherein the ratio of said THC: CBD is about 95:5% w/w; and
b. administering said composition to said subject orally in a therapeutically effective dosage form.

90. The method of claim 89, additionally comprising at least one step of
a. administering said composition in a dosage form comprising cannabinoids in a total amount of 2.5 mg to 50 mg per dosage form, preferably 5 mg per dosage form; or
b. administering said dosage form once or twice per day, for a period of 3 days to 6 months; or
c. treating a symptom associated with said syndrome, said symptom is selected from the group consisting of: weight loss, appetite loss, reduced caloric intake, elevated TNF-alpha level, anorexia, cachexia, reduced Quality-of-Life, mood related conditions, gastrointestinal problems, reduced muscle mass, pain, and any combination thereof; or
d. providing a beneficial effect selected from the group consisting of weight gain of at least 10% from baseline weight, improvement in appetite, improvement in caloric intake, reduction in TNF-alpha level, analgesic effects, antitumor activity, cancer cells cytotoxic effect, antidepressant, an anxiolytic, neuroprotective, anti-psychotic, improved quality of life, and any combination thereof; or
e. attenuating a symptom of said syndrome, treat said syndrome or attenuating a side effect of a treatment of said syndrome; or
f. administering said dosage form once per day for the first 3 to 4 days of the treatment; and twice per day from the 5th day of the treatment.

91. The method of claim 90, wherein at least one of the following holds true:
a. said improvement in quality of life is assessed using at least one of the European Organization of Research and Treatment of Cancer core questions on the Quality of Life Questionnaire, version 2 (QLQ-C30) and the Anorexia/Cachexia Therapy (FAACT) questionnaire; or
b. said method exerts reduced hallucinatory effects in the subject when compared to smoking a cannabis containing cigarette or ingesting a cannabis containing foodstuff with the same amount of active ingredient.

92. A process for the preparation of the composition of claim 76, comprising the steps of a. mixing a cannabinoid extract with an edible oil to formulate an immediate release (IR) formulation; b. filling a capsule with said IR formulation; c. freezing said filled capsule at −20° C.; d. thawing said freezed capsule; e. mixing a cannabinoid extract with at least one Lipid-based Drug Delivery System (LBDDS) agent and optionally an edible oil, to formulate sustained release (SR) formulation; and f adding said sustained release (SR) formulation to said thawed capsule.

93. The process of claim 92 further comprising at least one step of:
a. adding at least one of Monoglycerides, Diglycerides and Carrageenan to said mixture of step e); or
b. milling, drying, compressing or filling capsules, preferably filling capsules; or
c. mixing a cannabinoid extract comprising about 5 mg of cannabinoids.

94. A kit useful for treating cancer-related cachexia and anorexia syndrome (CACS) comprising:
a. a plurality of orally administrable dosage forms, wherein each dosage form comprising a lipophilic mixture of Cannabidiol (CBD) or derivative thereof—in an immediate release formulation and—Tetrahydrocannabinol (THC) in a Lipid-based Drug Delivery System (LBDDS) sustained-release formulation, further wherein the ratio of said THC:CBD is about 95:5% w/w; and
b. instructions for use of said dosage forms.

95. The kit of claim 94, wherein at least one of the following holds true:
a. said immediate release formulation comprises an edible oil and said sustained release formulation comprises at least one of at least one Lipid-based Drug Delivery System (LBDDS) agent, or an edible oil; or
b. the LBDDS agent is selected from the group consisting of a monoglyceride, a diglyceride, a carrageenan and any mixture thereof; or
c. said composition comprises cannabis extract in a relative amount range of about 0.01 V/V to about 0.02 V/V; or
d. said composition comprises Carrageenan in a relative amount of about 0.005 V/V; or
e. said composition comprises Mono and Diglyceride in a relative amount of about 0.075 V/V; or
f. said composition comprises coconut oil in a relative amount of about 0.92 V/V; or
g. further comprising at least one excipient selected from the group consisting of a diluent, a binder, a lubricant, a disintegrant, a flavoring agent, a coloring agent, a stabilizer, a surfactant, a glidant, a plasticizer, a preservative, an essential oil and a sweetener; or
h. the composition comprising cannabis extract in a relative amount of about 0.01 V/V, Carrageenan in a relative amount of about 0.005 V/V, Mono and Diglyceride in a relative amount of about 0.075 V/V and coconut oil in a relative amount of about 0.92 V/V; or
i. the cannabinoids are present in a total amount 2.5 mg to 50 mg per dosage form, preferably 5 mg per dosage form; or
j. said syndrome is associated with a symptom selected from the group consisting of: weight loss, appetite loss, reduced caloric intake, elevated TNF-alpha level, anorexia, cachexia, reduced Quality-of-Life, mood related conditions, gastrointestinal problems, reduced muscle mass, reduced muscle strength, pain, and any combination thereof; or
k. the composition is formulated as granules, powder, capsules, tablet, film, suspension, sachets, a chewing gum and suspension.
